SEQUENCE LISTING (8 sequences)

SEQ ID NO: 1 - protein, 383 aa, artificial sequence
  synthetic sequence
QGPPSLKESGPGLVAPSQSLSITCTVSGFSLTSYGVHWVRQPPGKGLEWIGVIWAGGSTNYNSALMSRLSISKDNSKSQVFLKMNSLQTDDTAMYYCAVYYGYLFFAYWGQGTLVTVSAAKTTPPSVYPLAPGSAAQTNSMVTLGCLVKGYFPEPVTVTWNSGSLSSGVHTFPAVLQSDLYTPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 213 aa, artificial sequence
  synthetic sequence
ENVLTQSPAIMSASPGEKVTMTCSASSSVSYMHWYQQKSTTSPKLWIYDTSKLASGVPGRFSGSGSGNSYSLTISSMEAEDVATYYCFQGSGYPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 10 aa, artificial sequence
  synthetic sequence
GFSLTSYGVH

SEQ ID NO: 4 - protein, 11 aa, artificial sequence
  synthetic sequence
VIWAGGSTNYN

SEQ ID NO: 5 - protein, 10 aa, artificial sequence
  synthetic sequence
VYYGYLFFAY

SEQ ID NO: 6 - protein, 10 aa, artificial sequence
  synthetic sequence
SASSSVSYMH

SEQ ID NO: 7 - protein, 7 aa, artificial sequence
  synthetic sequence
DTSKLAS

SEQ ID NO: 8 - protein, 10 aa, artificial sequence
  synthetic sequence
FQGSGYPLTF

(19) United States
(12) Patent Application Publication
(10) Pub. No.: US 2021/0213055 A1
(43) Pub. Date: Jul. 15, 2021

(54) COMPOSITIONS AND METHODS FOR INDUCING PHAGOCYTOSIS

(71) Applicant: The Board of Trustees of the Leland Stanford Junior University, Stanford, CA (US)

(72) Inventors: Irving L. Weissman, Stanford, CA (US); Amira A. Barkal, Stanford, CA (US)

(21) Appl. No.: 15/734,470

(22) PCT Filed: Jun. 12, 2019
(86) PCT No.: PCT/US2019/036793

§ 371 Date: Dec. 2, 2020

Related U.S. Application Data
(60) Provisional application No. 62/832,252, filed on Apr. 10, 2019.
(60) Provisional application No. 62/684,407, filed on Jun. 13, 2018.

(51) Int. Cl. A61K 35/15; A61K 39/395; C07K 16/28; C07K 16/30; G01N 33/574

(52) U.S. Cl. CPC A61K 35/15; A61K 39/3955; G01N 33/57492; C07K 16/2803; C07K 16/30; A61K 39/39558

(57) ABSTRACT

Methods and compositions are provided for inducing phagocytosis of a target cell in an individual, by blocking the interaction between CD24 on a target cell and Siglec10 on a phagocytic cell.

### CROSS REFERENCE

This application claims benefit of U.S. Provisional Patent Application No. 62/684,407, filed Jun. 13, 2018, U.S. Provisional Patent Application No. 62/832,252, filed Apr. 10, 2019, which applications are incorporated herein by reference in their entirety.

### FEDERALLY SPONSORED RESEARCH AND DEVELOPMENT

This invention was made with Government support under contracts CA220434 and CA232472 awarded by the National Institutes of Health. The Government has certain rights in the invention.

### INTRODUCTION

Programmed cell death (PCD) and phagocytic cell removal are common ways that an organism responds in order to remove damaged, precancerous, or infected cells. Cells, including although not limited to those undergoing apoptosis, have been found to have markers that target them appropriately for phagocytosis. These markers have been termed “eat-me” signals, which enhance phagocytosis, and “don't-eat-me” signals which can block or reduce phagocytosis. “Eat-me” signals prominently include exposed phosphatidylserine, which is recognized by a number of different receptors, and calreticulin bound to cell surface glycans. “Don't-eat-me” signals include, for example, the protein binding pairs of CD47/SIRPα; LILRB1/MHC Class I; and PD1/PDL1. Phagocytic cells express a number of receptors that may identify cells with these signals on their surface.

In some instances, cells such as cancer cells or infected cells co-opt the phagocytic control system by modifying expression of protein signals. Growing tumors and cells harboring an infection are under constant pressure from the host immune system, and evasion of immunosurveillance is critical for the progression of disease in patients. If properly engaged, phagocytic cells possess the ability to attack cancer cells and/or infected cells; and may further stimulate an adaptive immune response. For example, tumor-binding monoclonal antibodies can induce an attack, and efficacy is in part dependent on the antibody's ability to stimulate antibody-dependent cellular phagocytosis (ADCP) by macrophages.

However, CD47, a “don't eat me” signal, is constitutively upregulated on a wide variety of diseased cells, cancer cells, and infected cells, allowing these cells to evade phagocytosis. Although binding of an anti-tumor antibody to tumor cells is sufficient to engage macrophage Fc receptors and thereby stimulate some degree of tumor cell phagocytosis, the potency of this response is strongly limited by the tumor's expression of CD47. Therapeutic agents that disrupt this escape, either by directly stimulating the immune system to attack tumor cells and/or infected cells, or by blocking immunosuppressive signals expressed by tumor cells and/or infected cells, are a promising new category of drugs.

However, some cancer cells and/or infected cells are not fully susceptible to treatment with anti-CD47/SIRPA agents. The use of additional or alternative agents that are involved in the engagement of phagocytic cells is therefore of interest.

### PUBLICATIONS

Oldenborg P. A. et al. Role of CD47 as a marker of self on red blood cells. SCIENCE 288, 2051-2054 (2000). Jaiswal S. et al. CD47 is upregulated on circulating hematopoietic stem cells and leukemia cells to avoid phagocytosis. CELL 138, 271-285 (2009). Majeti R. et al. CD47 is an adverse prognostic factor and therapeutic antibody target on human acute myeloid leukemia stem cells. CELL 138, 286-299 (2009). Gordon S. G. et al. PD-1 expression by tumour-associated macrophages inhibits phagocytosis and tumour immunity. NATURE 545, 495-499 (2017). Barkal A. A. et al. Engagement of MHC class I by the inhibitory receptor LILRB1 suppresses macrophages and is a target of cancer immunotherapy. NATURE IMMUNOLOGY 19, 76-84 (2018). Advani R. et al. CD47 blockade by Hu5F9-G4 and rituximab in non-Hodgkin's lymphoma. NEW ENGLAND JOURNAL OF MEDICINE 379, 1711-1721 (2018). Chao, M. P. et al. Anti-CD47 antibody synergizes with rituximab to promote phagocytosis and eradicate non-Hodgkin lymphoma. CELL 142, 699-713 (2010). Willingham S. B. et al. The CD47-SIRPα interaction is a therapeutic target for human solid tumors. PROC. NATL. ACAD. SCI. USA 109, 6662-6667 (2012). Gholamin S. et al. Disrupting the CD47-SIRPα anti-phagocytic axis by a humanized anti-CD47 antibody is an efficacious treatment for malignant pediatric brain tumors. SCI. TRANSL. MED. 9, 381 (2017). Pirruccello S. J., LeBien T. W. The human B cell-associated antigen CD24 is a single chain sialoglycoprotein. J. IMMUNOL. 136, 3779-3784 (1986). Chen G. Y., Brown N. K., Zheng P., Liu Y. Siglec-G/10 in self-nonself discrimination of innate and adaptive immunity. GLYCOBIOLOGY 9, 800-806 (2014). Chen W. et al. Induction of Siglec-G by RNA viruses inhibits the innate immune response by promoting RIG-I degradation. CELL 152(3), 467-478 (2013). Chen G. Y. et al. Amelioration of sepsis by inhibiting sialidase-mediated disruption of the CD24-SiglecG interaction. NATURE BIOTECHNOLOGY 29, 428-435 (2011). Chen G. Y. et al. CD24 and Siglec-10 selectively repress tissue damage-induced immune responses. SCIENCE 323 (5922), 1722-1725 (2009). Toubai T. et al. Siglec-G-CD24 axis controls the severity of graft-versus-host disease in mice. BLOOD 123(22), 3512-3513 (2014). Crocker P. R., Paulson J. C., Varki A. NATURE REVIEWS IMMUNOLOGY. 7, 255-266 (2007). Abram C. L., Lowell C. A. Shp1 function in myeloid cells. J. LEUKOC. BIOL. 102(3), 657-675 (2017). An H. et al. Phosphatase SHP-1 promotes TLR- and RIG-I-activated production of type I interferon by inhibiting the kinase IRAK1. NATURE IMMUNOLOGY 9(5), 542-550 (2008). Zakia K. et al. Deficiency in Hematopoietic Phosphatase Ptpn6/Shp1 Hyperactivates the Innate Immune System and Impairs Control of Bacterial Infections in Zebrafish Embryos. J IMMUNOL 190(4), 1631-1645 (2013). Dietrich J., Cella M., Colonna M. Ig-Like Transcript 2 (ILT2)/Leukocyte Ig-Like Receptor 1 (LIR1) Inhibits TCR Signalling and Actin Cytoskeleton Reorganization. J. IMMUNOL. 166(4), 2514-2521 (2001). Tarhriz V. et al. Overview of CD24 as a new molecular marker in ovarian cancer. J. CELL. PHYSIOL. 234(3) 2134-2142 (2019). Kristiansen G. et al. CD24 expression is a new prognostic marker in breast cancer. CLIN. CANCER. RES. 15(9), 4906-4913 (2003). Kristiansen G. et al. CD24 is an independent prognostic marker of survival in non-small cell lung cancer patients. BR. J. CANCER, 88, 231-236 (2003). Kristiansen G. et al. CD24 expression is a significant predictor of PSA relapse and poor prognosis in low grade or organ confined prostate cancer. PROSTATE. 58(2), 183-192 (2004). Karaayvaz M. et al. Unravelling subclonal heterogeneity and aggressive disease states in TNBC through single-cell RNA-seq. NATURE COMMUNICATIONS 9, 3588 (2018). Mantovani A., Sozzani S., Locati M., Allavena P., Sica A. Macrophage polarization: tumor-associated macrophages as a paradigm for polarized M2 mononuclear phagocytes. TRENDS IN IMMUNOLOGY 23(11), 549-555 (2002). Liu J. et al. Pre-clinical development of a humanized anti-CD47 antibody with anti-cancer therapeutic potential. PLOS ONE 10, e0137345 (2015). Shultz L. D. et al. Human lymphoid and myeloid cell development in NOD/LtSz-scid IL2Ry null mice engrafted with mobilized human hemopoietic stem cells. J. IMMUNOL. 174, 6477-6489 (2005). Miksa M., Komura H., Wu R., Shah K. G., Wang P. A novel method to determine the engulfment of apoptotic cells by macrophages using pHrodo succinimidyl ester. J. IMMUNOL. Methods 342(1-2), 71-77 (2009). Okazaki M., Luo Yi., Han Tin., Yoshida M., Sean B. K. Three New Monoclonal Antibodies That Define a Unique Antigen Associated With Prolymphocytic Leukemia/Non-Hodgkin's Lymphoma and Are Effectively Internalized After Binding to the Cell Surface Antigen. BLOOD 81 (1), 84-94 (1993). Varga A. et al. Pembrolizumab in patients (pts) with PD-L1-positive (PD-L1+) advanced ovarian cancer: updated analysis of KEYNOTE-028. J. OLIN. ONCOL. 35 (abstract 5513) (2017). Infante J. R. et al. Safety, clinical activity and biomarkers of atezolizumab (atezo) in advanced ovarian cancer (OC). ANN. ONCOL. 27(6), 296-312 (2016). Nanda R. et al. Pebrolizumab in patients with advanced triple-negative breast cancer: Phase Ib KEYNOTE-012 study. J. CLIN. ONCOL. 34, 2460-2467 (2016). Daud, A. I. et al. Programmed death-ligand 1 expression and response to the anti-programmed death 1 antibody pembrolizumab in melanoma. J. OLIN. ONCOL. 34, 4102-4109 (2016). Reck M. et al. Pembrolizumab versus Chemotherapy for PD-L1-Positive Non-Small-Cell Lung Cancer. New England Journal of Medicine. 375(19) 1823-1833 (2016). Mittendorf E. A. et al. PD-L1 expression in triple-negative breast cancer. CANCER IMMUNOL. RES. 2, 361-370 (2014). Ali H. R. et al. PD-L1 protein expression in breast cancer is rare, enriched in basal-like tumours and associated with infiltrating lymphocytes. ANN. ONCOL. 26, 1488-1493 (2015). Ayers M. et al. Molecular Profiling of Cohorts of Tumor Samples to Guide Clinical Development of Pembrolizumab as Monotherapy. CLINICAL CANCER RES (2018). Noy R., Pollard J. W., Tumor-Associated Macrophages: From Mechanisms to Therapy. IMMUNITY 41(1), 49-61 (2014). Gentles A. J. et al. The prognostic landscape of genes and infiltrating immune cells across human cancers. NATURE MEDICINE 21(8), 938-945 (2015). Tseng D. et al. Anti-CD47 antibody-mediated phagocytosis of cancer by macrophages primes an effective antitumor T-cell response. PROC. NATL. ACAD. SCI. USA 110, 11103-11108 (2013). Anderson K. G., Stromnes I. M., and Greenberg P. D. Obstacles Posed by the Tumor Microenvironment to T cell Activity: A Case for Synergistic Therapies. CANCER CELL. 31(3), 311-325 (2017). Beatson R. et al. The mucin MUC1 modulates the tumor immunological microenvironment through engagement of the lectin Siglec-9. NATURE IMMUNOLOGY. 17(11), 1273-1281 (2016). Läubli H. et al. Engagement of myelomonocytic Siglecs by tumor-associated ligands modulates the innate immune response to cancer. PNAS 111(39), 14211-14216 (2014). Carlin A. F. et al. Group B Streptococcus suppression of phagocyte functions by protein-mediated engagement of human Siglec-5. J EXP MED. 206(8), 1691-1699 (2009). Ascierto M. L. et al. The intratumoral balance between metabolic and immunologic gene expression is associated with anti-PD-1 response in patients with renal cell carcinoma. CANCER IMMUNOL RES. 4(9), 726-733 (2016). Goldman M., Craft B., Brooks A. N., Zhu J., Haussler D. The USCS Xena Platform for cancer genomics data visualization and interpretation. BIORXIV (2018). Martinez, F. O. Analysis of Gene Expression and Gene Silencing in Human Macrophages. CURRENT PROTOCOLS IN IMMUNOLOGY. 96(1), 14.28.1-14.28.23 (2012). Brinkman E. K. et al. Easy quantitative assessment of genome editing by sequence trace decomposition. NUCLEIC ACIDS RESEARCH. 42(22), e168 (2014).

### SUMMARY

Methods and compositions are provided for inducing phagocytosis of a target cell, treating an individual having cancer, treating an individual having an intracellular pathogen infection, and/or reducing the number of inflicted cells (e.g., cancer cells, cells infected with an intracellular pathogen, etc.) in an individual. In some embodiments, phagocytosis is enhanced by contacting a target inflicted cell with a macrophage in the presence of an anti-CD24/Sialic acid-binding Ig-like lectin 10 (Siglec10) agent, which agent may include, without limitation, an antibody that specifically binds to CD24; an antibody that specifically binds to Siglec10; a soluble CD24 polypeptide; a soluble Siglec10 polypeptide. In some embodiments the anti-CD24/Siglec10 agent is administered in combination with an antibody that binds to the target cell, e.g. an antibody specific for a tumor cell antigen, an antibody specific for a pathogen antigen, etc. In some embodiments, the anti-CD24/Siglec10 agent is administered in combination with an additional phagocytosis enhancing therapy, including without limitation an agent that blocks the CD47/SIRPα; LILRB1/MHC Class I; or the PD1/PDL1 interaction. The target cells are contacted for a period of time sufficient to induce phagocytosis of the target cell by a phagocytic cell, e.g. a macrophage. In some cases, the contacting is in vitro or ex vivo. In some cases, the contacting is in vivo.

Methods and compositions are also provided for predicting whether an individual is resistant or susceptible to treatment with an agent that blocks the interaction between a “don't-eat-me” signal, e.g. CD47/SIRPα; LILRB1/MHC Class I; and PD1/PDL1. Cells that are determined to over-express CD24 relative to a control cell population are determined to be relatively resistant to phagocytosis, and may be treated with an anti-CD24/Siglec10 agent, administered in combination with an antibody that binds to the target cell; or with an additional phagocytosis enhancing therapy.

Kits are also provided for practicing the methods of the disclosure. In some embodiments, a kit composition for increasing phagocytosis of a target cell comprises: (a) an anti-CD24/Siglec10 agent (e.g., an CD24 binding agent such as an anti-CD24 antibody or an Siglec10 polypeptide; an Siglec10 binding agent such as an anti-Siglec10 antibody or a soluble CD24 polypeptide; and the like); and (b) at least one of: (i) an agent that opsonizes the target cell, e.g. a target cell specific antibody, and (ii) an agent other than CD24/Siglec10 agent that enhances phagocytosis. In some cases, the agent that opsonizes the target cell is an antibody other than an anti-CD47 antibody. In some cases, the composition includes an anti-CD47/SIRPA agent and an agent that opsonizes the target cell.

In some embodiments, a subject method is a method of treating an individual having cancer and/or having an intracellular pathogen infection where the method includes administering to the individual: (a) an anti-CD24/Siglec10 agent; and (b) at least one of: (i) an anti-CD47/SIRPA agent, and (ii) an agent that opsonizes a target cell of the individual, where the target cell is a cancer cell and/or a cell harboring an intracellular pathogen, in amounts effective for reducing the number of cancer cells and/or cells harboring the intracellular pathogen in the individual. In some cases, (a) and (b) are administered simultaneously. In some cases, (a) and (b) are not administered simultaneously. In some cases, the method includes, prior to the administering step: measuring the expression level of CD24 in a biological sample of the individual, where the biological sample includes a cancer cell and/or a cell harboring an intracellular pathogen; and providing a prediction, based on the result of the measuring step, that the individual is resistant to treatment with a phagocytosis enhancing agent other than CD24/Siglec10.

In some embodiments, a subject method is a method of predicting whether an individual is resistant or susceptible to treatment with a phagocytosis enhancing agent other than CD24/Siglec10, where the method includes: (a) measuring the expression level of CD24 in a biological sample of the individual, where the biological sample includes a cancer cell and/or a cell harboring an intracellular pathogen, to produce a measured test value; (b) comparing the measured test value to a control value; (c) providing a prediction; based on the comparing step, as to whether the individual is resistant or susceptible to treatment with a phagocytosis enhancing agent other than CD24/Siglec10, where increased expression of CD24 is indicative of resistance to a phagocytosis enhancing agent other than CD24/Siglec10; and (d) treating an individual in accordance with the prediction. An individual susceptible to treatment with a phagocytosis enhancing agent other than CD24/Siglec10 may be treated with an agent including, for example, blockade of CD47/Sirpα interaction. An individual resistant to treatment with a phagocytosis enhancing agent other than CD24/Siglec10 may be treated with a CD24/Siglec10 agent. In some cases, the measuring step includes an antibody-based method. In some cases, the antibody-based method includes flow cytometry. In some cases, the control value is the expression level of CD24 from a cell or population of cells known to exhibit a phenotype of resistance to treatment with an anti-CD47/SIRPA agent. In some cases, the control value is the background value of the measuring step. In some cases, the providing a prediction step includes generating a report that includes at least one of: (i) the measured expression level of CD24, (ii) the normalized measured expression level of CD24, (iii) a prediction of resistance or susceptibility to a phagocytosis enhancing agent other than CD24/Siglec10, and (iv) a recommended therapy based on the measured test value. In some cases, the report is displayed to an output device at a location remote to the computer. In some cases, a subject method includes a identifying/selecting a patient need of co-administration of an anti-CD24/Siglec10 agent and an additional phagocytosis enhancing agent.

In some embodiments a method for increasing phagocytosis, or compositions for use in such a method, utilize an anti-CD24 antibody. In some embodiments the antibody is a chimeric or humanized antibody comprising human Ig constant region sequences. In some embodiments the constant region is a gamma chain, for example selected from γ1, γ2a, γ2b, γ3, γ4 and derivatives thereof as known in the art. In some embodiments the anti-CD24 antibody comprises at least one, usually at least 3 CDR sequences from a set, as provided herein as SEQ ID NO:2, 3, 4 and SEQ ID NO:6, 7, 8, usually in combination with framework sequences from a human variable region. In some embodiments an antibody comprises at least one light chain comprising a set of 3 light chain CDR sequences provided herein situated in a variable region framework, which may be, without limitation, a human or mouse variable region framework, and at least one heavy chain comprising the set of 3 heavy chain CDR sequence provided herein situated in a variable region framework, which may be, without limitation, a human or mouse variable region framework. In other embodiments, the antibody comprises an amino acid sequence variant of one or more of the CDRs of the provided antibodies, which variant comprises one or more amino acid insertion(s) within or adjacent to a CDR residue and/or deletion(s) within or adjacent to a CDR residue and/or substitution(s) of CDR residue(s) (with substitution(s) being the preferred type of amino acid alteration for generating such variants). Such variants will normally having a binding affinity for human CD26 of at least about 10⁻⁸ M and will bind to the same epitope as an antibody having the amino acid sequence of those set forth herein. The antibody may be a full length antibody, e.g. having a human immunoglobulin constant region of any isotype, e.g. IgG1, IgG2a, IgG2b, IgG3, IgG4, IgA, etc. or an antibody fragment, e.g. a F(ab′)₂ fragment, and F(ab) fragment, etc. Furthermore, the antibody may be labeled with a detectable label, immobilized on a solid phase and/or conjugated with a heterologous compound.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1F. CD24 expression is upregulated in human cancers and is an adverse prognostic indicator. FIG. 1A Heat map of log₂ fold change (Log₂FC) in normalized expression values of all ligands for ITIM-bearing macrophage receptors collected in 27 human cancers from The Cancer Genome Atlas (TCGA) and TARGET, versus TOGA or GTEX matched normal tissues. CD24 log₂FC is highlighted in the green box. Data were normalized and compiled by UCSC Xena. FIG. 1B Scatter plot of log₂FC of CD47 expression vs. log₂FC of CD24 expression in 27 human cancers. Ovarian carcinoma (OV, red box), breast carcinoma (BRCA, blue box), lung squamous cell carcinoma (LUSC, green box), lung adenocarcinoma (LUAD, yellow box), acute myeloid leukemia (LAML, purple box). FIG. 1C, 1E Scatter plot CD24 mRNA expression (log₂(TPM+1)) from TOGA primary human ovarian carcinoma samples (FIG. 1C) or breast carcinoma samples (FIG. 1E) versus matched healthy tissues, ****=p<0.0001. FIG. 1D, 1F Kaplan-Meier plots demonstrating overall survival of patients with high CD24 expression (red) or low CD24 expression (blue) in (FIG. 1D) ovarian carcinoma (n=352 low; n=1044 high) or (FIG. 1F) breast carcinoma (n=1955 low, n=1965 high). Survival data was collected from Km Plotter (Lanczky et al., Breast Cancer Res Treat. 2016).

FIG. 2. CD24 knockout promotes the phagocytosis of MCF7 breast cancer cells. CD24−/− MCF7 cells are more susceptible to phagocytosis by macrophages both in the absence (left) and presence (right) of CD47 blockade.

FIG. 3A-3B. Siglec-10 blockade promotes the phagocytosis of MCF7 breast cancer cells and primary human ovarian carcinoma cells. FIG. 3A Siglec-10 monoclonal antibodies (Clone 1D11; Novus Bio) promote the phagocytosis of CD24+ MCF7 cells and FIG. 3B primary ovarian carcinoma cells in vitro both in the presence and absence of CD47 blockade.

FIG. 4A-4C. CD24 blocking antibodies promote the phagocytosis of human cancers. CD24 monoclonal antibodies (Clone SN3; Thermofisher Scientific) promote the phagocytosis of CD24+ FIG. 4A MCF7 breast cancer, FIG. 4B NCI-H82 small cell lung cancer (SCLC), and FIG. 4C primary ovarian carcinoma cells as compared to isotype controls.

FIG. 5A-5H. CD24 is over-expressed by human cancers and is co-expressed with Siglec-10 on TAMs (FIG. 5A), Heatmap of tumor to matched normal expression ratios (log 2FC) for CD24 as compared to known innate immune checkpoint molecules, CD47, PD-L1, and B2M (tumor study abbreviations and n defined in Extended Data Table 1). FIG. 5A, Expression levels of CD24 in ovarian cancer (red boxplot, n=419) in comparison with ovarian tissue from healthy individuals (gray boxplot, n=89), ****P<0.0001, unpaired, two-tailed Student's t-test. FIG. 5C, Expression levels of CD24 in TNBC (red boxplot, n=124) in comparison with ER+PR+ breast cancer (purple boxplot, n=508) and normal breast (gray boxplot, n=293). Each symbol represents an individual patient sample. ****P<0.0001, one-way ANOVA with multiple comparisons correction, F_{(2,922)}=95.80. FIG. 5D, Kaplan-Meier relapse-free survival curves for ovarian cancer patients with high versus low CD24 expression (log 2(TPM+1) as defined by median CD24 expression. P value computed by a log-rank (Mantel-Cox) test. FIG. 5E, Kaplan-Meier Overall Survival curves for breast cancer patients with high versus low CD24 expression (log 2(TPM)+1) as defined by median CD24 expression. P value computed by a log-rank (Mantel-Cox) test. FIG. 5F, UMAP dimension 1 and 2 plots displaying all TNBC cells analyzed from 6 patients=1001 single cells); (left) cell clusters are differentially colored by cell class as defined by marker gene expression, (right) expression of CD24 (red) and Siglec-10 (blue) overlaid onto the UMAP space as compared to CD47 expression (gray) and PD-L1 expression (gray) (labels on plots; arrow pointing to Tumor cluster in CD24 panel, arrow pointing to TAM cluster in Siglec-10 panel). FIG. 5G, (left) Representative flow cytometry histogram measuring the expression of CD24 (red shaded curves) versus isotype control (black lines) by primary ovarian cancer cells (top) or primary breast cancer cells (bottom), numbers above bracketed line indicate percent cancer cells positive for expression of CD24; (right) frequency of primary human cancer cells positive for CD24 among total EpCAM+ tumor cells as defined by isotype controls (mean±s.e.m) in primary ovarian cancer (n=3 donors) (top) or primary breast cancer (n=5 donors) (bottom). FIG. 5H, (left) Representative flow cytometry histogram measuring the expression of Siglec-10 (blue shaded curves) versus isotype control (black lines) by primary ovarian cancer TAMs (top) or primary breast cancer TAMs (bottom), numbers above bracketed line indicate percent TAMs positive for expression of Siglec-10; (right) frequency of primary human TAMs positive for Siglec-10 among TAMs as defined by isotype controls (mean±s.e.m) in primary ovarian cancer (n=6 donors) (top) or primary breast cancer=5 donors) (bottom).

FIG. 6A-6M. CD24 directly protects cancer cells from phagocytosis by macrophages FIG. 6A, Schematic depicting interactions between macrophage-expressed Siglec-10 (blue) and CD24 expressed by cancer cells (red). FIG. 6B, Flow cytometry-based measurement of the surface expression of CD24 on MCF-7 cells (blue shaded curve) versus CD24 knockout cells (ΔCD24) (red shaded curve) as compared to isotype control (black line), numbers above bracketed line indicate percent MCF-7 WT cells positive for expression of CD24. FIG. 6C, (left) Flow cytometry-based measurement of the surface expression of Siglec-10 on primary human donor-derived macrophages either unstimulated (top) or following stimulation with M2-polarizing cytokines TGFβ1 and IL-10 (bottom), numbers above bracketed line indicate percent CD11b+ macrophages positive for expression of Siglec-10; (right) Frequency of primary human donor-derived macrophages positive for Siglec-10 either without stimulation (Unstimulated, M0) or following stimulation with TGFβ1 and IL-10 (Stimulated, M2-like), (paired, two-tailed Student's t-test with multiple comparisons correction, ****P<0.0001). FIG. 6D, Flow cytometry-based measurement of phagocytosis of CD24+ parental MCF-7 cells (WT) and CD24− (ΔCD24) MCF-7 cells by cocultured human macrophages, in the presence or absence of anti-CD47 mAb (horizontal axis), (n=4 donors; two-way ANOVA with multiple comparisons correction, cell line F_{(1,12)}=65.65; treatment F_{(1,12)}=40.30, **P<0.01, ****P<0.0001). FIG. 6E, Representative images from live-cell microscopy phagocytosis assays of pHrodo-red-labeled, GFP+ MCF-7 cells (WT, top; ΔCD24, bottom); phagocytosis is depicted by increased red signal indicative of engulfment into low pH phagolysosome and decreased green signal due to phagocytic clearance of GFP+ cells over time (hours elapsed listed beneath images); images are representative of two biological donors and technical replicates. FIG. 6F, Flow cytometry-based measurement of in vivo phagocytosis of CD24₊GFP₊ID8 cells (WT) versus CD24-GFP₊ ID8 cells (ΔCd24a) by mouse peritoneal macrophages, (unpaired, two-tailed Student's t-test with multiple comparisons correction, *P<0.05). FIG. 6G, Flow cytometry-based measurement of phagocytosis of parental MCF-7 cells by co-cultured human macrophages, in the presence of anti-Siglec10 mAb or IgG control (n=4 donors; paired, one-tailed Student's t-test, ***P<0.001). FIG. 6H, Flow cytometry-based measurement of binding of recombinant Siglec10-Fc to MCF-7 WT cells treated with neuraminidase (green shaded curve, +NA) or heat-inactived neuraminidase (+HI-NA, blue shaded curve); plot is representative of two experimental replicates. FIG. 6I, (left) Flow cytometry-based measurement of binding of Siglec10-Fc to neuraminidase-treated MCF-7 WT cells (blue shaded curve) vs. neuraminidase-treated MCF-7ΔCD24 cells, same voltage as in i; (right) normalized binding of Siglec10-Fc to MCF-7ΔCD24 cells (red bar) as computed as the fraction of Siglec10-Fc+ cells among total MCF-7ΔCD24 cells divided by the percentage of Siglec10-Fc+ cells among total MCF-7 WT cells (blue bar) (n=two experimental replicates with polyclonal ΔCD24 sublines and 2 polyclonal WT lines; unpaired, two-tailed Student's t-test, **P<0.01). FIG. 6J, (left) Flow cytometry-based measurement of the surface expression of Siglec-10 by Siglec10 KO donorderived macrophages (red shaded curve) vs. Cas9 control (blue shaded curve) 72 h postelectroporation; (right) Frequency of primary human donor-derived macrophages positive for Siglec-10 among Cas9 control macrophages (blue dots) vs. Siglec-10 KO macrophages (red dots), (n=5 biological donors; data are from two experimental replicates). FIG. 6K, Flow cytometry-based measurement of phagocytosis of parental MCF-7 cells by either Siglec-10 KO macrophages (red) or Cas9 control macrophages (blue), (n=5 biological donors; data are from two experimental replicates; paired; one-tailed Student's t-test, **P<0.01). FIG. 6L, Representative images from live-cell microscopy phagocytosis assays of pHrodo-red-labeled MCF-7 cells treated with anti-CD24 mAb (right) or IgG control (left) at t=5:05 h; images are representative of two biological donors and four technical replicates per donor. FIG. 6M, Quantification of phagocytosis events of MCF-7 cells treated with anti-CD24 mAb (red curve) versus IgG control (blue curve) as measured by live-cell microscopy over time in hours (h), normalized to maximum measured phagocytosis events per donor, (n=two biological donors, 4 technical replicates per donor; P value computed by two-way ANOVA, F_{(1,24)}=65.02). Data are mean±s.e.m.

FIG. 7A-7G. Treatment with anti-CD24 mAb promotes phagocytic clearance of human cancer cells FIG. 7A Representative flow cytometry plots depicting phagocytosis of MCF-7 cells treated with anti-CD24 mAb, CD47 mAb, or dual treatment with anti-CD24 mAb and anti-CD47 mAb, as compared to IgG control. Plots are representative of 5 independent donors each assayed in technical triplicate. Numbers indicate frequency of phagocytosis events (CD11b+F1TC+) out of total macrophages (CD11b+). FIG. 7B, Flow cytometry-based measurement of phagocytosis of the CD24+ cell lines MCF-7 (n=5 donors), APL1 (n=8 donors), and Panc 1 (n=8 donors) (left) and the CD24− U87-GM cell line (n=3 donors: solid bars) (right) by donor-derived macrophages in the presence of anti-CD24 mAb, anti-CD47 mAb or both anti-CD24 mAb and anti-CD47 mAb, as compared to IgG control (treatments listed below plot); each symbol represents an individual donor (one-way ANOVA with multiple comparisons correction; MCF-7 F_{(3,16)}=145.6, APL1 F_{(3,28)}=144.7, Panc1 F_{(3,28)}=220.7, U-87 MG F_{(3,8)}=200.4; NS=not significant, **P<0.01, ***P<0.001, ****P<0.0001). FIG. 7C Response to anti-CD24 mAb as computed by the phagocytosis fold change between CD24 mAb treatment and IgG control by donor-derived macrophages stimulated with TGFβ1 and IL-10 (M2-like) vs. unstimulated (M0); each symbol represents an individual donor (paired, two-tailed Student's r-test, *P<0.05) FIG. 7D, Response to anti-CD24 mAb as computed by the phagocytosis fold change between CD24 mAb treatment and IgG control by donor-derived Siglec10 knockout macrophages (un-shaded dots) vs. donor-matched macrophages which received Cas9 alone (blue dots); (n=4 donors, connecting lines indicate matched donor. Paired, one-tailed Student's t-test, **P<0.01) FIG. 7E, Correlation between cancer cell CD24 expression (MFI=median fluorescence intensity) (x-axis) and response to anti-CD24 mAb as computed by the phagocytosis fold change between anti-CD24 mAb treatment and IgG control (y-axis). FIG. 7F, Workflow to purify primary ovarian cancer cells from ascites fluid and co-culture with donor-derived macrophages in the presence of anti-CD24 mAb to measure phagocytosis, FIG. 7G, Flow cytometry-based measurement of phagocytosis of primary ovarian cancer cells in the presence of anti-CD24 mAb, anti-CD47 mAb, or both anti-CD24 mAb and anti-CD47 mAb, as compared to IgG control (n=10 macrophage donors challenged with n=1 primary ovarian cancer ascites donor) (one-way ANOVA with multiple comparisons correction, F_{(2.110, 18.99)}=121.5, **P<0.01, ***P<0.001, ****P<0.0001). Data are mean±s.e.m.

FIG. 8A-8G. CD24 protects cancer cells from macrophage attack in vivo FIG. 8A, Representative flow cytometry plots demonstrating TAM phagocytosis in GFP-luciferase+ CD24+(WT) MCF-7 tumors (left) vs. CD24− (ΔCD24) MCF-7 tumors (middle), numbers indicate frequency of phagocytosis events out of all TAMs; (right) frequency of phagocytosis events out of all TAMs in WT tumors vs. ΔCD24 tumors 28 days after engraftment (WT n=10, ΔCD24 n=9. Unpaired, two-tailed Student's t-test). FIG. 8B, Representative bioluminescence image of tumor burden in NSG mice engrafted with MCF-7 WT vs. MCF-7ΔCD24 tumors (image taken 21 days post-engraftment). FIG. 8C, Burden of MCF-7 WT tumors (blue) vs. MCF-7ΔCD24 tumors (red) in mice either treated with TAMs (vehicle, shaded circles) or mice depleted of TAMs (unshaded squares) as measured by bioluminescence imaging (Two-way ANOVA with multiple comparisons correction, tumor genotype F_{(3,33)}=11.75). FIG. 8D, Survival analysis of vehicle-treated mice in c, P value computed by a log-rank (Mantel-Cox) test (WT n=5, ΔCD24 n=5). e, Burden of MCF-7 WT tumors treated with IgG control (blue) vs. anti-CD24 mAb (red) as measured by bioluminescence (IgG control n=10, anti-CD24 mAb n=10. Two-way ANOVA with multiple comparisons correction, tumor treatment F_{(1; 126)}=5.679). FIG. 8E, Burden of ID8 WT tumors (blue) vs. ID8ΔCd24a tumors (red) as measured by bioluminescence imaging (WT n=5, ΔCd24a n=5. Two-way ANOVA with multiple comparisons correction, tumor genotype F_{(1,48)}=10.70).

FIG. 8F, Burden of MCF-7 WT tumors treated with IgG control (blue) vs. anti-CD24 mAb (red) as measured by bioluminescence (IgG control n=10, anti-CD24 mAb n=10. Days on which anti-CD24 mAb was administered are indicated by arrows below x-axis. Data are of two independent experimental cohorts. Two-way ANOVA with multiple comparisons correction, tumor treatment F_{(1,126)}=5.679). FIG. 8G, Representative bioluminescence image of tumor burden in NSG mice with MCF-7 tumors treated with either IgG control or anti-CD24 mAb (image taken 33 days post-engraftment). *P<0.05, ***P<0.001, ****P<0.0001. Data are mean±s.e.m.

FIG. 9A-9D. Expression of innate immune checkpoints in human cancer FIG. 9A, Heatmap of expression (log 2(Normalized counts+1)) of CD24 from bulk TOGA/TARGET studies, as compared to known innate immune checkpoint molecules, CD47, PD-L1, and B2M (tumor study abbreviations and n defined in Table 1). FIG. 9B, Heatmap of marker gene expression (y-axis) across TNBC single cells (x-axis) and cell clusters identified (top). FIG. 9C, UMAP dimension 1 and 2 plots displaying all TNBC cells analyzed from six patients (n=1001 single cells); cell clusters are colored by cell patient (key, left). FIG. 9D, CD24 vs. PD-L1 expression in the “Tumor epithelial cell” cluster for individual TNBC patients; number of single cells analyzed, PT039 n=151 cells, PT058 n=11 cells, PT081 n=196 cells, PT084 n=84 cells, PT089 n=117, PT126 n=60 cells. **P<0.01, ****P<0.0001. Data are violin plots showing median expression (log 2(Norm counts+1) and quartiles (paired, two-tailed t-test).

FIG. 10A-10F. Flow-cytometry analysis of CD24 and Siglec-10 expression in human tumors and primary immune cells FIG. 10A, Gating strategy for CD24⁺ cancer cells and Siglec-10⁺ TAMs in primary human tumors; after debris and doublet removal, cancer cells were assessed as DAPI-CD14− EpCAM⁺ and TAMs were assessed as DAPI-EpCAM-CD14⁺CD11b⁺. FIG. 10B, (top) Representative flow cytometry histogram measuring the expression of Siglec-10 (blue shaded curves) versus isotype control (black lines) by non-cancerous peritoneal macrophages, numbers above bracketed line indicate percent macrophages positive for expression of Siglec-10; (bottom) frequency of peritoneal macrophages positive for Siglec-10 among all peritoneal macrophages as defined by isotype controls (n=9 donors). FIG. 10C, Gating strategy for CD24+ cells and Siglec-10+ cells among PBMC cell types; after debris and doublet removal, monocytes were assessed as DAPI-CD3-CD14⁺; T cells were assessed as DAPI-CD14-CD3+; NK cells were assessed as DAPI-CD14-CD3-CD56⁺; B cells were assessed as DAPI-CD56-CD14-CD3-CD19⁺. FIG. 10D, Frequency of PBMC cell types positive for Siglec-10 (blue shaded bars) or CD24 (red shaded bars) out of total cell type (cell type assessed labeled on top of individual plots). FIG. 10E, Flow cytometry-based measurement of phagocytosis of MCF-7 cells by unstimulated donor-derived macrophages (white dots) versus TGFβ-1 and IL-10-stimulated donor-derived macrophages (n=3 donors, unpaired, one-tailed ttest, *p<0.05). FIG. 10F, (left) Flow cytometry-based measurement of the surface expression of Siglec-10 on matched, primary donor-derived macrophages either unstimulated (gray shaded curve), or following stimulation with TGFβ1 and IL-10 (blue line), or IL-4 (green line); (right) Frequency of matched, human donor-derived macrophages positive for Siglec-10 either without stimulation (unstimulated, M0), or following stimulation with TGFβ1 and IL-10 (blue dots), or stimulated with IL-4. Data are mean±s.e.m.

FIG. 11A-11G, Siglec-10 binds to CD24 expressed on MCF-7 cells FIG. 11A, Flow cytometry histogram measuring binding of Siglec-10 to WT MCF-7 cells (blue shaded curve) versus ΔCD24 MCF-7 cells (red shaded curve). Data are representative of two experimental replicates. FIG. 11B, Merged flow cytometry histogram measuring binding of Siglec-10-Fc to WT MCF-7 cells treated with heat-inactivated neuraminidase (WT-HI NA, blue line), WT MCF-7 cells treated with neuraminidase (WT-NA, green line), ΔCD24 MCF-7 cells treated with heat-inactivated neuraminidase (red line, ΔCD24-HI NA), and ΔCD24 MCF-7 cells treated with neuraminidase (purple line, ΔCD24-NA) as compared to isotype control (black line). Data are representative of two experimental replicates. FIG. 11C, Flow cytometry-based measurement of phagocytosis of CD24+ parental MCF-7 cells (WT) and CD24− (ΔCD24) MCF-7 cells by cocultured human macrophages in the presence of neuraminidase (+NA) or heat-inactivated neuraminidase (+HI-NA)=4 donors; two-way ANOVA with multiple comparison's correction, cell line F_{(1,12)}=180.5, treatment F_{(1,12)}=71.12, ****P<0.0001). FIG. 11D,11F Representative flow cytometry histogram measuring the binding of Siglec-5, FIG. 11D, or Siglec-9, FIG. 11F, to WT MCF-7 cells treated with either vehicle (blue shaded curve) or neuraminidase (green shaded curve). Data are representative of two experimental replicates. FIG. 11E, 11G, (left) Representative flow cytometry histogram measuring the expression of Siglec-5, FIG. 11E, or Siglec-9, FIG. 11G, (blue shaded curves) versus isotype control (black lines) by stimulated (M2-like) macrophages; (right) frequency of macrophages positive for Siglec-5, FIG. 11D, or Siglec-9, FIG. 11F, among unstimulated M0 macrophages (white dots) or stimulated M2-like macrophages (blue dots) (n=9 donors).

FIG. 12. Gating strategy for in vitro phagocytosis assay. Following debris and doublet removal, phagocytosis was assessed as the frequency of DAPI-CD11b⁺FITC⁺ events out of all DAPI-CD11b⁺ events. Numbers indicate frequency of events out of previous gate.

FIG. 13A-13J. CD24 antibody blockade of CD24-Siglec-10 signaling promotes dose-responsive enhancement of phagocytosis FIG. 13A, Schematic of CD24-Siglec-10 inhibition of phagocytosis; the inhibitory receptor Siglec-10 engages its ligand CD24 on cancer cells, leading to phosphorylation of the two ITIM motifs in the cytoplasmic domain of Siglec-10 and subsequent anti-inflammatory, anti-phagocytic signaling cascades mediated by SHP-1 and SHP-2 phosphatases; upon the addition of a CD24 blocking antibody, macrophages are disinhibited and thus capable of phagocytosis-mediated tumor clearance. FIG. 13B, Dose-response relationship of anti-CD24 mAb on phagocytosis of MCF-7 cells, concentrations listed on the x-axis as compared to IgG control. FIG. 13C, Flow cytometry-based measurement of phagocytosis of NCI-H82 cells by donor-derived macrophages (n=3 donors) in the presence of anti-CD24 mAb as compared to IgG control; each symbol represents an individual donor (paired, two-tailed Student's t-test). FIG. 13D, Flow cytometry-based measurement of phagocytosis of CD24+ parental MCF-7 cells (WT) and CD47− (ΔCD47) MCF-7 cells by cocultured human macrophages, in the presence or absence of anti-CD24 mAb (horizontal axis), (n=4 donors; two-way ANOVA with multiple comparisons correction, cell line F_{(1,8)}=6.490; treatment F_{(1,8)}=98.73, **P<0.01). FIG. 13E, Flow cytometry-based measurement of phagocytosis of Panc1 pancreatic adenocarcinoma cells in the presence of anti-CD24 mAb, cetuximab (anti-EGFR), or both anti-CD24 mAb and cetuximab, as compared to IgG control (n=6 donors) (one way ANOVA with multiple comparisons correction, F_{(3,20)}=66.10. *P<0.05, **P<0.01. Data are mean±s.e.m. FIG. 13F, (left) Representative flow cytometry histogram measuring the expression of EpCAM (green shaded curve) by parental MCF-7 cells, number above bracketed line indicates percent MCF-7 cells positive for expression of EpCAM; (right) Flow cytometry-based measurement of phagocytosis of parental MCF-7 cells by co-cultured human macrophages, in the presence of either IgG control, anti-EpCAM mAb, or anti-CD24 mAb (n=4 donors: repeated measures ANOVA with multiple comparisons correction, F_{(2,9)}=340.9, *P<0.05, **P<0.01, ****P<0.0001). FIG. 13G, Correlation between stimulated (M2-like) donor-derived macrophage Siglec-10 expression (MFI=median fluorescence intensity) (x-axis) and response to anti-CD24 mAb as computed by the phagocytosis fold change between anti-CD24 mAb treatment and IgG control (y-axis), (n=7 donors); exponential growth curve is shown. FIG. 13H, Fold change in phagocytosis by M0 (unstimulated) or M2-like (TGFβ-1, IL-10-stimulated) macrophages upon the addition of anti-EpCAM mAb as compared to IgG control, (n=9 donors. Paired, two-tailed t-test, NS=not significant). FIG. 13I, Correlation between cancer cell CD24 expression (MFI=median fluorescence intensity) (x-axis) and baseline, un-normalized phagocytosis levels (IgG control) averaged across all donors per cell line. Exponential growth equation is shown. FIG. 13J, Flow cytometry-based measurement of phagocytosis of a patient sample of primary TNBC cells in the presence of anti-CD24 mAb, anti-CD47 mAb, or both anti-CD24 mAb and anti-CD47 mAb, as compared to IgG control (n=3 macrophage donors challenged with n=1 primary TNBC donor) (repeated measures one-way ANOVA with multiple comparisons correction, F_{(1.217,2.434)}=26.17). Each point represents an individual donor. NS=not significant, *P<0.05, **P<0.01, ***P<0.001. Data are mean±s.e.m.

FIG. 14A-14D. Characterization of MCF-7 WT and MCF-7ΔCD24 cells in vitro and in vivo FIG. 14A, Gating strategy for in vivo TAM phagocytosis of MCF-7 cells: following debris and doublet removal, TAM phagocytosis assessed as the frequency of DAPI-CD11b⁺F4/80⁺GFP⁺ events out of total DAPI-CD11b⁺F4/80⁺ events; M1-like TAMs assessed as DAPI-CD11b⁺F4/80⁺CD80⁺, Numbers indicate frequency of events out of previous gate. FIG. 14B, Frequency of TAMs positive for CD80 (M1-like) as per gating in a, among all TAMs macrophages as defined by fluorescence minus one controls (WT n=10, ΔCD24 n=9). *P<0.05. Data are mean±s.e.m. FIG. 14C, In vitro proliferation rates of MCF-7 WT and MCF-7ΔCD24 as assessed by confluence percentage (y axis) over time (x-axis), (n=6 technical replicates). FIG. 14D, (top) Representative flow cytometry histogram of the surface expression of CD24 on Day 35 WT MCF-7 tumors (blue shaded curve) versus Day 35 CD24 knockout tumors (ΔCD24) (red shaded curve) as compared to isotype control (black line), see FIG. 2b for Day 0 measurement of CD24 expression; (bottom) flow cytometry-based measurement of the frequency of CD24+ cells among all cancer cells in Day 35 WT tumors versus Day 35 ΔCD24 tumors (WT n=4, ΔCD24 n=4). Data are mean±s.e.m.

FIG. 15. Depletion of tissue-resident macrophages by anti-CSF1R mAb. Representative flow cytometry plots of tissue-resident macrophages out of total live cells in vehicle-treated animals (left) vs. anti-CSF1R-treated animals (middle), numbers indicate frequency of CD11b+,F4/80+ macrophage events out of total live events; (right) frequency of TAMs (CD11b+,F4/80+) out of total live cells in vehicle-treated animals (n=5, blue shaded boxplot) vs. anti-CSF1R-treated animals (n=4, red shaded boxplot) as measured by flow cytometry. **p<0.01. Boxplots depict mean and range.

FIG. 16A-16B. Validation of CD24 inhibition in in vivo models of ovarian and breast cancer FIG. 16A, Representative bioluminescence image of tumor burden in C57Bl/6 mice with ID8 WT vs. ID8ΔCd24a tumors (image taken 49 days post-engraftment). FIG. 16A, Extended measurement of burden of MCF-7 WT tumors treated with IgG control (blue) vs. anti-CD24 mAb (red) as measured by bioluminescence (IgG control n=5, anti-CD24 mAb n=5. Days on which anti-CD24 mAb was administered are indicated by arrows below x-axis. Data are of one independent experimental cohort. Two-way ANOVA with multiple comparisons correction, tumor treatment F_{(1,81)}=16.75). ****P<0.0001. Data are mean±s.e.m.

FIG. 17A-17C. Anti-CD24 mAb induces B cell clearance but does not bind human RBCs, and CD47 and CD24 subset human DLBCL demonstrating inversely correlated expression FIG. 17A, Flow cytometry-based measurement of phagocytosis of B cells (n=4 donors, pooled) by donor-derived macrophages (n=4 donors) in the presence of anti-CD24 mAb as compared to IgG control; each symbol represents an individual donor (paired, two-tailed Student's t-test). FIG. 17B, (left) Representative flow cytometry histogram measuring the expression of CD24 (red line) and CD47 (blue line) by human RBCs; (right) Flow-cytometry-based measurement of the frequency of CD24⁺ versus CD47⁺ RBCs out of total RBCs (n=3 donors). FIG. 17C, (left) Expression levels in log₂(norm counts+1) of CD24 and CD47 in Diffuse Large B Cell Lymphomas from TCGA (n=48), data are divided into quadrants by median expression of each gene as indicated by dotted lines, number and percentage of total patients in each quadrant indicated on plot. Each dot indicates a single patient; (right) 2-dimensional contour plot of Diffuse Large B Cell Lymphoma patients in left plot. Data are mean±s.e.m.

### DETAILED DESCRIPTION

Methods and compositions are provided for inducing phagocytosis of a target cell, treating an individual having cancer, treating an individual having an intracellular pathogen infection, and/or reducing the number of inflicted cells, e.g. cancer cells, cells infected with an intracellular pathogen, etc. in an individual. Methods and compositions are also provided for predicting whether an individual is resistant (or susceptible) to treatment with a phagocytosis enhancing agent other than CD24/Siglec10. In some cases, the subject methods and compositions comprise an anti-CD24/Siglec10 agent. In some cases, the subject methods and compositions comprise an anti-CD24/Siglec10 agent and an agent that opsonizes a target cell. In some cases, the subject methods and compositions comprise an anti-CD24/Siglec10 agent and an anti-CD47/SIRPA agent, which may be co-administered. Kits are also provided for practicing the methods of the disclosure.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

In the description that follows, a number of terms conventionally used in the field are utilized. In order to provide a clear and consistent understanding of the specification and claims, and the scope to be given to such terms, the following definitions are provided.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, aspirate, and the like. A “biological sample” includes a sample comprising target cells and/or normal control cells, or is suspected of comprising such cells. The definition includes biological fluids derived therefrom (e.g., cancerous cell, infected cell, etc.), e.g., a sample comprising polynucleotides and/or polypeptides that is obtained from such cells (e.g., a cell lysate or other cell extract comprising polynucleotides and/or polypeptides). A biological sample comprising an inflicted cell (e.g., cancer cell, an infected cell, etc.) from a patient can also include non-inflicted cells.

The term “diagnosis” is used herein to refer to the identification of a molecular or pathological state, disease or condition, such as the identification of a molecular subtype of cancer, the determination that an individual is resistant or susceptible to treatment with a phagocytosis enhancing agent other than CD24/Siglec10, and the like.

The term “prognosis” is used herein to refer to the prediction of the likelihood of disease progression (e.g., cancer-attributable death or progression, progression of an infection, etc.), including recurrence, metastatic spread of cancer, and drug resistance.

The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence. As another example, one may predict the likelihood that an individual is resistant (or susceptible) to treatment with a phagocytosis enhancing agent other than CD24/Siglec10. As an example, one may predict the likelihood that an individual is susceptible to treatment with an anti-CD47/SIRPA agent.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides/epitopes). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻ⁱⁱ M or less, 10⁻¹² M or less, 10⁻¹³ M or less, 10⁻¹⁴ M or less, 10⁻¹⁵ M or less, or 10⁻′⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Examples of specific binding members include, but are not limited to: agents that specifically bind CD24, Siglec10, LILRB1, MHC Class I, CD47, and/or SIRPα (i.e., anti-CD24/Siglec10 agents, anti-CD47/SIRPα agents), or that otherwise block the interaction between CD24 and Siglec10; and/or the interaction between CD47 and SIRPα.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species. Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region.

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or phagocytosis. A functional antibody may also block ligand activation of a receptor or act as an agonist or antagonist.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein may comprise any suitable Fc region, including without limitation, human or other mammalian, e.g. cynomogulus, IgG, IgE, IgM, IgD, IgA, IgG1, IgG2, IgG3, IgG4, IgA1 and IgA2 or subclass of immunoglobulin molecule, including engineered subclasses with altered Fc portions that provide for reduced or enhanced effector cell activity. The immunoglobulins can be derived from any species.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include at least one CDR sequence from the variable regions provided herein, usually at least 2 CDR sequences, and more usually 3 CDR sequences. exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139, each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1 IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

A “functional Fc region” possesses an “effector function” of a native-sequence Fc region. Exemplary effector functions include C1q binding; CDC; Fc-receptor binding; ADCC; ADCP; down-regulation of cell-surface receptors (e.g., B-cell receptor), etc. Such effector functions generally require the Fc region to be interact with a receptor, e.g. the FcγRI; FcγRIIA; FcγRIIB1; FcγRIIB2; FcγRIIIA; FcγRIIIB receptors, and the law affinity FcRn receptor; and can be assessed using various assays as disclosed, for example, in definitions herein. A “dead” Fc is one that has been mutagenized to retain activity with respect to, for example, prolonging serum half-life; but which does not activate a high affinity Fc receptor.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). Other Fc variants are possible, including without limitation one in which a region capable of forming a disulfide bond is deleted, or in which certain amino acid residues are eliminated at the N-terminal end of a native Fc form or a methionine residue is added thereto. Thus, in one embodiment of the invention, one or more Fc portions of the scFc molecule can comprise one or more mutations in the hinge region to eliminate disulfide bonding. In yet another embodiment, the hinge region of an Fc can be removed entirely. In still another embodiment, the molecule can comprise an Fc variant.

Further, an Fc variant can be constructed to remove or substantially reduce effector functions by substituting, deleting or adding amino acid residues to effect complement binding or Fc receptor binding. For example, and not limitation, a deletion may occur in a complement-binding site, such as a C1q-binding site. Techniques of preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms; e.g. Pichia pastoris, and mammalians cell line, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem.; 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the function of Immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See e.g., Kaneko, et al, Science 313:760 (2006)), whereas removal of fucose from the IgG leads to enhanced ADCC activity (See e.g., Shoj-Hosaka, et al, J. Biochem., 140:777 (2006)).

In alternative embodiments, antibodies of the invention may have an Fc sequence with enhanced effector functions, e.g. by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into>50-fold higher ADCC activity compared with wild type IgG1 controls. Protein engineering, through amino acid mutations in the Fc portion of IgG1, has generated multiple variants that increase the affinity of Fc binding to FcγRIIIA. Notably; the triple alanine mutant S298A/E333A/K334A displays 2-fold increase binding to FcγRIIIA and ADCC function. S239D/I332E (2×) and S239D/I332E/A330L (3×) variants have a significant increase in binding affinity to FcγRIIIA and augmentation of ADCC capacity in vitro and in vivo. Other Fc variants identified by yeast display also showed the improved binding to FcγRIIIA and enhanced tumor cell killing in mouse xenograft models. See, for example Liu et al. (2014) JBC 289(6):3571-90, herein specifically incorporated by reference.

“Fv” is the minimum antibody fragment, which contains a complete antigen-recognition and antigen-binding site. The Fab fragment contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; nanobodies comprising single Ig domains from non-human species or other specific single-domain binding modules; and multispecific or multivalent structures formed from antibody fragments.

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The word “label” when used herein refers to a detectable compound or composition which is conjugated directly or indirectly, e.g., to a subject anti-CD24/Siglec10 agent and/or anti-CD47/SIRPA agent. The label may itself be detectable by itself (directly detectable label) (e.g., radioisotope labels or fluorescent labels) or, or the label can be indirectly detectable, e.g., in the case of an enzymatic label, the enzyme may catalyze a chemical alteration of a substrate compound or composition and the product of the reaction is detectable.

The terms “phagocytic cells” and “phagocytes” are used interchangeably herein to refer to a cell that is capable of phagocytosis. There are four main categories of phagocytes: macrophages, mononuclear cells (histiocytes and monocytes); polymorphonuclear leukocytes (neutrophils) and dendritic cells. In some embodiments a phagocytic cell is a macrophage.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

### Compositions

The present disclosure provides compositions for enhancing phagocytosis of a target cell, treating an individual having cancer, treating an individual having an intracellular pathogen infection (e.g., a chronic infection), reducing the number of inflicted cells (e.g., cancer cells, cells infected with an intracellular pathogen, etc.) in an individual, and/or predicting whether an individual is resistant (or susceptible) to treatment with an anti-CD47/SIRPA agent. In some cases, the subject compositions include an anti-CD24/Siglec10 agent. In some cases, the subject compositions include an anti-CD24/Siglec10 agent and an anti-CD47/SIRPA agent.

Anti-CD24/Siglec10 agent. CD24 is a two-chain glycosylphosphatidylinositol (GPI)-anchored glycoprotein expressed at multiple stages of B-cell development, beginning with the bone marrow pro-B-cell compartment and continuing through mature, surface Ig positive B-cells. Plasma cell expression is very low or negative. It is also expressed on the majority of B-lineage acute lymphoblastic leukemias, B-cell CCLs and B-cell non-Hodgkin's lymphomas. CD24 may play a role in regulation of B-cell proliferation and maturation. Protein references sequences include Genbank NP_001278666; NP_001278667; NP_001278668; NP_037362; NP 001346013. Antibodies known to bind to human CD24 are known and commercially available, including, without limitation, MA5-11833; 12-0247-42; anti-CD24 clone ML5 (Biolegend), SN3 A5-2H10 (also referred to as SN3); etc. An anti-CD24 agent may include, for example, an antibody that binds to human CD24, such as SN3.

Sialic acid-binding Ig-like lectin 10. SIGLECs are members of the immunoglobulin superfamily that are expressed on the cell surface. Most SIGLECs have 1 or more cytoplasmic immune receptor tyrosine-based inhibitory motifs, or ITIMs. SIGLECs are typically expressed on cells of the innate immune system. Siglec10 is a ligand for CD52, VAP-1 and CD24. Reference sequences for Siglec10 protein from Genbank include NP_766488, NP_001164628, NP_001164629, NP_001164630, NP_001164632. Antibodies specific for the human protein are known and commercially available, for example 1D11, 5G6, etc.

An CD24 protein on a first cell (e.g., a cancer cell, an infected cell) can bind to (and activate) Siglec10 on a second cell (e.g., a phagocytic cell, e.g., a macrophage) and thereby inhibit phagocytosis of the first cell by the second cell. When “activated,” the receptor transduces a negative signal that inhibits stimulation of an immune response in the cells on which it is expressed.

As used herein, the term “anti-CD24/Siglec10 agent” refers to any agent that reduces the binding of CD24 (e.g., on a target cell) to Siglec10 (e.g., on a phagocytic cell). An anti-CD24 agent binds to CD24, e.g. an anti-CD24 antibody, or a soluble Siglec10 polypeptide. An anti-Siglec10 agent binds to Siglec10, e.g. an anti-Siglec10 antibody, or a soluble CD24 polypeptide.

In some embodiments, a suitable anti-CD24/Siglec10 agent (e.g. an anti-CD24 antibody, a Siglec10 peptide, etc.) specifically binds CD24 and reduces the binding of CD24 to Siglec10. In some embodiments, a suitable anti-CD24/Siglec10 agent (e.g. an anti-CD24 antibody, a Siglec10 peptide, etc.) specifically binds CD24 and reduces the binding of CD24 to Siglec10. In some cases, an anti-CD24/Siglec10 agent (e.g., in any of the methods or compositions of the disclosure) is an antibody, and in some cases it is a humanized antibody. Small molecule compounds that inhibit the binding of CD24 with Siglec10 are also considered to be anti-CD24/Siglec10 agents. Anti-CD24/Siglec10 agents do not activate/stimulate Siglec10 on the Siglec10-expressing phagocytic cell. In some cases, anti-CD24/Siglec10 agents do not activate/stimulate Siglec10 to an amount where signaling via Siglec10 is stimulated on phagocytic cells, thereby inhibiting phagocytosis by the phagocytic cells. In other words, in some cases, a suitable anti-CD24/Siglec10 agent that binds Siglec10 can stimulate some level of signaling via Siglec10 on phagocytic cells, as long as the level of signaling is not enough to inhibit phagocytosis.

The efficacy of a suitable anti-CD24/Siglec10 agent can be assessed by assaying the agent. As a non-limiting example of such an assay, target cells are incubated in the presence or absence of the candidate agent, and phagocytosis of the target cells is measured (e.g., phagocytosis by macrophages). An agent for use in the subject methods (an anti-CD24/Siglec10 agent) will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, at least 200%, or at least 300%) compared to phagocytosis in the absence of the candidate agent. Any convenient phagocytosis assay can be used. As a non-limiting example of a phagocytosis assay, see the Examples below.

In some cases, the assay can be conducted in the presence of a known phagocytosis inducing agent (e.g., an anti-CD47/SIRPA agent). In some cases, in the presence of a known phagocytosis inducing agent (e.g., an anti-CD47/SIRPA agent), an anti-CD24/Siglec10 agent will up-regulate regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, at least 200%, or at least 300%) compared to phagocytosis in the absence of the phagocytosis inducing agent. In some cases, in the presence of a known phagocytosis inducing agent (e.g., an anti-CD47/SIRPA agent), an anti-CD24/Siglec10 agent will up-regulate regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, at least 200%, or at least 300%) compared to phagocytosis in the absence of the candidate agent.

Anti-CD47/SIRPA agent. As used herein, the term “anti-CD47/SIRPA agent” refers to any agent that reduces the binding of CD47, e.g., on a target cell, to SIRPA (also known as SIRPα), e.g., on a phagocytic cell. Non-limiting examples of suitable anti-CD47/SIRPA agents include SIRPA reagents, including without limitation high affinity SIRPA polypeptides; anti-SIRPA antibodies; soluble CD47 polypeptides; and anti-CD47 antibodies or antibody fragments. In some embodiments, a suitable anti-CD47/SIRPA agent (e.g. an anti-CD47 antibody, a SIRPA reagent, etc.) specifically binds CD47 to reduce the binding of CD47 to SIRPA. In some embodiments, a suitable anti-CD47/SIRPA agent (e.g., an anti-SIRPA antibody, a soluble CD47 polypeptide, etc.) specifically binds SIRPA to reduce the binding of CD47 to SIRPA. A suitable anti-CD47/SIRPA agent that binds SIRPA does not activate SIRPA (e.g., in the SIRPA-expressing phagocytic cell). The efficacy of a suitable anti-CD47/SIRPA agent can be assessed by assaying the agent (further described below). As a non-limiting example of such an assay, target cells are incubated in the presence or absence of the candidate agent, and phagocytosis of the target cells is measured (e.g., phagocytosis by macrophages). An agent for use in the subject methods (an anti-CD47/SIRPA agent) will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, at least 200%, or at least 300%) compared to phagocytosis in the absence of the candidate agent. Any convenient phagocytosis assay can be used. As a non-limiting example of a phagocytosis assay, see the Examples below. Similarly, an in vitro assay that measures tyrosine phosphorylation of SIRPA can be used (e.g., as an alternative or in addition to a phagocytosis assay). A suitable candidate agent will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47/SIRPA agent does not activate CD47 upon binding. When CD47 is activated, a process akin to apoptosis (i.e., programmed cell death) may occur (Manna and Frazier, Cancer Research, 64, 1026-1036, Feb. 1 2004). Thus, in some embodiments, the anti-CD47/SIRPA agent does not directly induce cell death of a CD47-expressing cell.

SIRPA reagent. A SIRPA reagent comprises the portion of SIRPA that is sufficient to bind CD47 at a recognizable affinity, which normally lies between the signal sequence and the transmembrane domain, or a fragment thereof that retains the binding activity. A suitable SIRPA reagent reduces (e.g., blocks, prevents, etc.) the interaction between the native proteins SIRPA and CD47. The SIRPA reagent will usually comprise at least the dl domain of SIRPA. In some embodiments, a SIRPA reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPA reagent. In other embodiments, the second polypeptide is any suitable polypeptide that is substantially similar to Fc, e.g., providing increased size, multimerization domains, and/or additional binding or interaction with Ig molecules.

In some embodiments, a subject anti-CD47/SIRPA agent is a “high affinity SIRPA reagent”, which includes SIRPA-derived polypeptides and analogs thereof. High affinity SIRPA reagents are described in international application PCT/US13/21937, which is hereby specifically incorporated by reference. High affinity SIRPA reagents are variants of the native SIRPA protein. In some embodiments, a high affinity SIRPA reagent is soluble, where the polypeptide lacks the SIRPA transmembrane domain and comprises at least one amino acid change relative to the wild-type SIRPA sequence, and wherein the amino acid change increases the affinity of the SIRPA polypeptide binding to CD47, for example by decreasing the off-rate by at least 10-fold, at least 20-fold, at least 50-fold, at least 100-fold, at least 500-fold, or more.

A high affinity SIRPA reagent comprises the portion of SIRPA that is sufficient to bind CD47 at a recognizable affinity, e.g., high affinity, which normally lies between the signal sequence and the transmembrane domain, or a fragment thereof that retains the binding activity. The high affinity SIRPA reagent will usually comprise at least the dl domain of SIRPA with modified amino acid residues to increase affinity. In some embodiments, a SIRPA variant of the present invention is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPA reagent. In other embodiments, the second polypeptide is any suitable polypeptide that is substantially similar to Fc, e.g., providing increased size, multimerization domains, and/or additional binding or interaction with Ig molecules. The amino acid changes that provide for increased affinity are localized in the dl domain, and thus high affinity SIRPA reagents comprise a dl domain of human SIRPA, with at least one amino acid change relative to the wild-type sequence within the dl domain. Such a high affinity SIRPA reagent optionally comprises additional amino acid sequences, for example antibody Fc sequences; portions of the wild-type human SIRPA protein other than the dl domain, including without limitation residues 150 to 374 of the native protein or fragments thereof, usually fragments contiguous with the dl domain; and the like. High affinity SIRPA reagents may be monomeric or multimeric, i.e. dimer, trimer, tetramer, etc. An example of a high-affinity SIRPA reagent is known as CV1 (an engineered protein monomer).

Anti-CD47 antibodies. In some embodiments, a subject anti-CD47/SIRPA agent is an antibody that specifically binds CD47 (i.e., an anti-CD47 antibody) and reduces the interaction between CD47 on one cell (e.g., an infected cell) and SIRPA on another cell (e.g., a phagocytic cell). In some embodiments, a suitable anti-CD47 antibody does not activate CD47 upon binding. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies (e.g., hu5F9-G4) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Anti-SIRPA antibodies. In some embodiments, a subject anti-CD47/SIRPA agent is an antibody that specifically binds SIRPA (i.e., an anti-SIRPA antibody) and reduces the interaction between CD47 on one cell (e.g., an infected cell) and SIRPA on another cell (e.g., a phagocytic cell). Suitable anti-SIRPA antibodies can bind SIRPA without activating or stimulating signaling through SIRPA because activation of SIRPA would inhibit phagocytosis. Instead, suitable anti-SIRPA antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPA antibody specifically binds SIRPA (without activating/stimulating enough of a signaling response to inhibit phagocytosis) and blocks an interaction between SIRPA and CD47. Suitable anti-SIRPA antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Soluble CD47 polypeptides. In some embodiments, a subject anti-CD47/SIRPA agent is a soluble CD47 polypeptide that specifically binds SIRPA and reduces the interaction between CD47 on one cell (e.g., an infected cell) and SIRPA on another cell (e.g., a phagocytic cell). A suitable soluble CD47 polypeptide can bind SIRPA without activating or stimulating signaling through SIRPA because activation of SIRPA would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPA without activating/stimulating enough of a signaling response to inhibit phagocytosis.

In some cases, a suitable soluble CD47 polypeptide can be a fusion protein (for example as structurally described in US Patent Publication US20100239579, herein specifically incorporated by reference). However, only fusion proteins that do not activate/stimulate SIRPA are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPA and inhibit the interaction between CD47 and SIRPA without stimulating enough SIRPA activity to inhibit phagocytosis. In certain embodiments, soluble CD47 polypeptide comprises the extracellular domain of CD47, including the signal peptide. Soluble CD47 polypeptides also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPA without stimulating SIRPA signaling.

The above described agents can be prepared in a variety of ways. For example, an anti-CD24/Siglec10 agent and/or anti-CD47/SIRPA agent can be prepared (together or separately): as a dosage unit, with a pharmaceutically acceptable excipient, with pharmaceutically acceptable salts and esters, etc. Compositions can be provided as pharmaceutical compositions.

Pharmaceutical Compositions. Suitable anti-CD24/Siglec10 agents and/or anti-CD47/SIRPA agents can be provided in pharmaceutical compositions suitable for therapeutic use, e.g. for human treatment. In some embodiments, pharmaceutical compositions of the present invention include one or more therapeutic entities of the present disclosure (e.g., an anti-CD24/Siglec10 agent and/or an anti-CD47/SIRPA agent) and include a pharmaceutically acceptable carrier, a pharmaceutically acceptable salt, a pharmaceutically acceptable excipient, and/or esters or solvates thereof. In some embodiments, the use of an anti-CD24/Siglec10 agent and/or anti-CD47/SIRPA agent includes use in combination with another therapeutic agent (e.g., another anti-infection agent or another anti-cancer agent). Therapeutic formulations comprising an anti-CD24/Siglec10 agent and/or an anti-CD47/SIRPA agent can be prepared by mixing the agent(s) having the desired degree of purity with a physiologically acceptable carrier, a pharmaceutically acceptable salt, an excipient, and/or a stabilizer (Remington's Pharmaceutical Sciences 16th edition, ©sol, A. Ed. (1980)) (e.g., in the form of lyophilized formulations or aqueous solutions). A composition having an anti-CD24/Siglec10 agent and/or an anti-CD47/SIRPA agent can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

### Methods

Methods are provided for inducing phagocytosis of a target cell, treating an individual having cancer, treating an individual having an intracellular pathogen infection (e.g., a chronic infection), reducing the number of inflicted cells (e.g., cancer cells, cells infected with an intracellular pathogen, etc.) in an individual, and/or predicting whether an individual is resistant (or susceptible) to treatment with an anti-CD47/SIRPA agent. In some cases, the subject methods include the use of an anti-CD24/Siglec10 agent and an agent that opsonizes a target cell (e.g., co-administration of an anti-CD24/Siglec10 agent and an agent that opsonizes a target cell). In some cases, the subject methods include the use of an anti-CD24/Siglec10 agent and an anti-CD47/SIRPA agent (e.g., co-administration of an anti-CD24/Siglec10 agent and an anti-CD47/SIRPA agent). In some cases, the subject methods include the use of an anti-CD24/Siglec10 agent, an anti-CD47/SIRPA agent, and an agent that opsonizes a target cell (e.g., co-administration of an anti-CD24/Siglec10 agent, an anti-CD47/SIRPA agent, and an agent that opsonizes a target cell). In some cases an anti-CD47/SIRPA agent is an agent that opsonizes a target cell (e.g., when the anti-CD47/SIRPA agent is an anti-CD47 antibody having an Fc region).

The compositions described above can find use in the methods described herein.

In some cases, a subject method is a method of inducing phagocytosis of a target cell. The term “target cell” as used herein refers to a cell (e.g., inflicted cells such as cancer cells, infected cells, etc.) that is targeted for phagocytosis by a phagocytic cell. In some cases, a target cell is resistant to treatment with an anti-CD47/SIRPA agent. For example, some inflicted cells (e.g., cancer cells) do not express CD24 and such cells are predicted to be susceptible to an anti-CD47/SIRPA agent. When a target cell that is susceptible to an anti-CD47/SIRPA agent is contacted with a phagocytic cell in the presence of an anti-CD47/SIRPA agent, the target cell can be engulfed (e.g., phagocytosed) by the phagocytic cell.

However, some inflicted cells (e.g., cancer cells) do express CD24 and such cells may be resistant to an anti-CD47/SIRPA agent. When a target cell that is resistant to an anti-CD47/SIRPA agent is contacted with a phagocytic cell (e.g., a macrophage) in the presence of an anti-CD47/SIRPA agent, the target cell is less likely to be phagocytosed by the phagocytic cell. In some embodiments, a target cell is contacted with a phagocytic cell (e.g., a macrophage) in the presence of an anti-CD24/Siglec10 agent and an anti-CD47/SIRPA agent. When a target cell that is resistant to an anti-CD47/SIRPA agent (e.g., the resistant target cell expresses CD24) is contacted with a phagocytic cell (e.g., a macrophage) in the presence of an anti-CD24/Siglec10 agent and an anti-CD47/SIRPA agent, the phagocytic cell can engulf the target cell. Contacting a target cell with a phagocytic cell (e.g., a macrophage) in the presence of an anti-CD24/Siglec10 agent and an anti-CD47/SIRPA agent encompasses scenarios where the target cell is contacted with the anti-CD24/Siglec10 agent and the anti-CD47/SIRPA agent at the same time (i.e, both agents are present at the same time), and scenarios where the target cell is contacted with one of the agents prior to the other agent (in either order)(e.g., one of the agents is present first, and the other agent is later added, either in the presence or absence of the first agent).

Contacting a target cell with a phagocytic cell (e.g., a macrophage) in the present of an anti-CD24/Siglec10 agent and an anti-CD47/SIRPA agent can occur in vitro or in vivo. For example, in some cases, a target cell (e.g., a cancer cell from an individual, a cancer cell of an immortalized cell line, an infected cell from an individual, an infected cell of a cell line, and the like) is cultured in vitro with a phagocytic cell, an anti-CD24/Siglec10 agent, and an anti-CD47/SIRPA agent.

In some cases, after the phagocytic cell engulfs the target cell, the phagocytic cell is introduced into an individual (e.g., the individual from whom the target cell was taken). In some cases, the phagocytic cell is a cell from an individual (e.g., the same individual from whom the target cell was taken) and the phagocytic cell is re-introduce into the individual after the phagocytic cell engulfs the target cell. When the target cell and/or the phagocytic cell is from an individual that is being treated, the method can be referred to as an ex vivo method. In some cases, a method of inducing phagocytosis of a target cell, where the method includes contacting the target cell with a phagocytic cell (e.g., a macrophage) in the presence of an anti-CD24/Siglec10 agent and an anti-CD47/SIRPA agent, can occur in vivo. In such cases, the anti-CD24/Siglec10 agent and the anti-CD47/SIRPA agent can be administered to an individual (e.g., an individual having cancer, a chronic infection, etc.) and the contact of the target cell with the phagocytic cell will happen in vivo, without further input from the one performing the method. As such, in some cases, a method of inducing phagocytosis of a target cell can encompass a method that includes administering to an individual an anti-CD24/Siglec10 agent and an anti-CD47/SIRPA agent.

A target cell may be a cell that is “inflicted”, where the term “inflicted” is used herein to refer to a subject with symptoms, an illness, or a disease that can be treated with an anti-CD24/Siglec10 agent and an anti-CD47/SIRPA agent. An “inflicted” subject can have cancer, can harbor an infection (e.g., a chronic infection), and other hyper-proliferative conditions, for example sclerosis, fibrosis, and the like, etc. “Inflicted cells” may be those cells that cause the symptoms, illness, or disease. As non-limiting examples, the inflicted cells of an inflicted patient can be cancer cells, infected cells, and the like. One indication that an illness or disease can be treated with an anti-CD47/SIRPA agent is that the involved cells (i.e., the inflicted cells, e.g., the cancerous cells, the infected cells, etc.) express an increased level of CD47 compared to normal cells of the same cell type. One indication that an illness or disease can be treated with an anti-CD24/Siglec10 agent is that the involved cells (i.e., the inflicted cells, e.g., the cancerous cells, the infected cells, etc.) express CD24. In some cases, an indication that an illness or disease can be treated with an anti-CD24/Siglec10 agent and an anti-CD47/SIRPA agent is that the involved cells (i.e., the inflicted cells, e.g., the cancerous cells, the infected cells, etc.) express an increased level of CD47 compared to normal cells of the same cell type, and express CD24.

In some cases, a subject method is a method of treating an individual having cancer and/or having an intracellular pathogen infection (e.g., a chronic infection). An effective treatment will reduce the number of inflicted cells (e.g., cancer cells, cells infected with an intracellular pathogen, etc.) in an individual (e.g., via increasing phagocytosis of the target cells). As such, in some cases, a subject method is a method of reducing the number of inflicted cells (e.g., cancer cells, cells infected with an intracellular pathogen, etc.) in an individual.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, those with an immune disorder, etc.) as well as those in which prevention is desired (e.g., those with increased susceptibility to cancer, those with an increased likelihood of infection, those suspected of having cancer, those suspected of harboring an infection, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

Examples of symptoms, illnesses, and/or diseases that can be treated with an anti-CD24/Siglec10 agent (e.g. in combination with an anti-CD47/SIRPA agent or an opsonizing agent) include, but are not limited to cancer (any form of cancer, including but not limited to: carcinomas, soft tissue tumors, sarcomas, teratomas, melanomas, leukemias, lymphomas, brain cancers, solid tumors, mesothelioma (MSTO), etc.); infection from an intracellular pathogen (e.g., chronic infection); and immunological diseases or disorders (e.g., an inflammatory disease)(e.g., multiple sclerosis, arthritis, and the like)(e.g., for immunosuppressive therapy).

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Hematopoietic malignancies are leukemias, lymphomas and myelomas. Leukemias are cancers that start in blood-forming tissue, such as the bone marrow, and causes large numbers of abnormal blood cells to be produced and enter the bloodstream. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL. The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma); chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

As used herein, the term “infection” refers to any state in at least one cell of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen) (e.g., one that induces increased CD47 expression in at least one cell of the infected organism). For example, infectious agents include, but are not limited to bacteria, viruses, protozoans, and fungi. Intracellular pathogens are also of interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

Infectious diseases that can be treated using a subject anti-CD24/Siglec10 agent and/or anti-CD47/SIRPA agent include but are not limited to: HIV, Influenza, Herpes, Giardia, Malaria, Leishmania, the pathogenic infection by the virus Hepatitis (A, B, & C), herpes virus (e.g., VZV, HSV-I, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus, pathogenic infection by the bacteria chlamydia, rickettsial bacteria, mycobacteria, staphylococci, streptococci, pneumonococci, meningococci and conococci, klebsiella, proteus, serratia, pseudomonas, E. coli, legionella, diphtheria, salmonella, bacilli, cholera, tetanus, botulism, anthrax, plague, leptospirosis, and Lyme's disease bacteria, pathogenic infection by the fungi Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum, and pathogenic infection by the parasites Entamoeba histolytica, Balantidium coli, Naegieriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and/or Nippostrongylus brasiliensis.

In some embodiments the infliction is a chronic infection, i.e. an infection that is not cleared by the host immune system within a period of up to 1 week, 2 weeks, etc. In some cases, chronic infections involve integration of pathogen genetic elements into the host genome, e.g. retroviruses, lentiviruses, Hepatitis B virus, etc. In other cases, chronic infections, for example certain intracellular bacteria or protozoan pathogens, result from a pathogen cell residing within a host cell. Additionally, in some embodiments, the infection is in a latent stage, as with herpes viruses or human papilloma viruses.

An infection treated with the methods of the invention generally involves a pathogen with at least a portion of its life-cycle within a host cell, i.e. an intracellular phase. The methods of the invention provide for a more effective removal of infected cells by the phagocytic cells of the host organism, relative to phagocytosis in the absence of treatment, and thus are directed to the intracellular phase of the pathogen life cycle.

The terms “co-administration”, “co-administer”, and in combination with include the administration of two or more therapeutic agents (e.g., an anti-CD24/Siglec10 agent and an anti-CD47/SIRPA agent and/or a target cell specific antibody) either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

In some cases, a subject an anti-CD24/Siglec10 agent optionally combined with an anti-CD47/SIRPA agent (e.g., formulated as a pharmaceutical composition) is co-administered with a cancer therapeutic drug, therapeutic drug to treat an infection, or tumor-directed antibody. Such administration may involve concurrent (i.e. at the same time), prior, or subsequent administration of the drug/antibody with respect to the administration of an agent or agents of the disclosure. A person of ordinary skill in the art would have no difficulty determining the appropriate timing, sequence and dosages of administration for particular drugs and compositions of the present disclosure.

In some embodiments, treatment is accomplished by administering a combination (co-administration) of a subject anti-CD24/Siglec10 agent (e.g., with or without an anti-CD47/SIRPA agent) with another agent (e.g., an immune stimulant, an agent to treat chronic infection, a cytotoxic agent, an anti-cancer agent, etc.). One example class of cytotoxic agents that can be used are chemotherapeutic agents. Exemplary chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine and vinorelbine tartrate.

An anti-CD24/Siglec10 agent need not be, but is optionally formulated with one or more agents that potentiate activity, or that otherwise increase the therapeutic effect. These are generally used in the same dosages and with administration routes as used herein or from 1 to 99% of the heretofore employed dosages. In some embodiments, treatment is accomplished by administering a combination (co-administration) of a subject anti-CD24/Siglec10 agent and an agent that opsonizes a target cell. In some embodiments, treatment is accomplished by administering a combination (co-administration) of a subject anti-CD24/Siglec10 agent, an agent that opsonizes a target cell, and an anti-CD47/SIRPA agent. In some embodiments, treatment is accomplished by administering a combination (co-administration) of a subject anti-CD24/Siglec10 agent and an anti-CD47/SIRPA agent. Thus, also envisioned herein are compositions (and methods that use the compositions) that include: (a) an anti-CD24/Siglec10 agent; and (b) at least one of: (i) an agent that opsonizes the target cell, and (ii) an anti-CD47/SIRPA agent.

An “agent that opsonizes a target cell” (an “opsonizing agent”) is any agent that can bind to a target cell (e.g., a cancer cell, a cell harboring an intracellular pathogen, etc.) and opsonize the target cell. For example, any antibody that can bind to a target cell (as defined herein), where the antibody has an FC region, is considered to be an agent that opsonizes a target cell. In some cases, the agent that opsonizes a target cell is an antibody, other than an anti-CD47 antibody, that binds to a target cell (e.g., an anti-tumor antibody, an anti-cancer antibody, an anti-infection antibody, and the like).

For example antibodies selective for tumor cell markers, radiation, surgery, and/or hormone deprivation, see Kwon et al., Proc. Natl. Acad. Sci U.S.A., 96: 15074-9, 1999. Angiogenesis inhibitors can also be combined with the methods of the invention. A number of antibodies are currently in clinical use for the treatment of cancer, and others are in varying stages of clinical development. For example, there are a number of antigens and corresponding monoclonal antibodies for the treatment of B cell malignancies. One target antigen is CD20. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Two new monoclonal antibodies targeting CD20, tositumomab and ibritumomab, have been submitted to the Food and Drug Administration (FDA). These antibodies are conjugated with radioisotopes. Alemtuzumab (Campath) is used in the treatment of chronic lymphocytic leukemia; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

Monoclonal antibodies useful in the methods of the invention that have been used in solid tumors include, without limitation, edrecolomab and trastuzumab (herceptin). Edrecolomab targets the 17-1A antigen seen in colon and rectal cancer, and has been approved for use in Europe for these indications. Trastuzumab targets the HER-2/neu antigen. This antigen is seen on 25% to 35% of breast cancers. Cetuximab (Erbitux) is also of interest for use in the methods of the invention. The antibody binds to the EGF receptor (EGFR), and has been used in the treatment of solid tumors including colon cancer and squamous cell carcinoma of the head and neck (SCCHN).

A subject anti-CD24/Siglec10 agent can be combined (with or without an anti-CD47/SIRPA agent) any of the above mentioned antibodies (agents that opsonize a target cell). Thus, in some cases, a subject anti-CD24/Siglec10 agent is used in a combination therapy (is co-administered) with one or more cell-specific antibodies selective for tumor cell markers. in some cases, a subject anti-CD24/Siglec10 agent, is used in a combination therapy (is co-administered) with an anti-CD47/SIRPA agent and one or more cell-specific antibodies selective for tumor cell markers.

In some cases, a subject anti-CD24/Siglec10 agent, is used in a combination therapy (is co-administered) with one or more of: cetuximab (binds EGFR), panitumumab (binds EGFR), rituximab (binds CD20), trastuzumab (binds HER2), pertuzumab (binds HER2), alemtuzumab (binds CD52), brentuximab (binds CD30), tositumomab, ibritumomab, gemtuzumab, ibritumomab, and edrecolomab (binds 17-1A).

In some cases, a subject anti-CD24/Siglec10 agent, is used in a combination therapy (is co-administered) with an anti-CD47/SIRPA agent and one or more of: cetuximab (binds EGFR), panitumumab (binds EGFR), rituximab (binds CD20), trastuzumab (binds HER2), pertuzumab (binds HER2), alemtuzumab (binds CD52), brentuximab (binds CD30), tositumomab, ibritumomab, gemtuzumab, ibritumomab, and edrecolomab (binds 17-1A).

In some cases, a subject anti-CD24/Siglec10 agent is used in a combination therapy (is co-administered) with one or more agents that specifically bind one or more of: CD19, CD20, CD22, CD24, CD25, CD30, CD33, CD38, CD44, CD52, CD56, CD70, CD96, CD97, CD99, CD123, CD279 (PD-1), CD274 (PD-L1), EGFR, 17-1A, HER2, CD117, C-Met, PTHR2, and HAVCR2 (TIM3).

In some cases, a subject anti-CD24/Siglec10 agent is used in a combination therapy (is co-administered) with an anti-CD47/SIRPA agent and one or more agents that specifically bind one or more of: CD19, CD20, CD22, CD24, CD25, CD30, CD33, CD38, CD44, CD52, CD56, CD70, CD96, CD97, CD99, CD123, CD279 (PD-1), CD274 (PD-L1), EGFR, 17-1A, HER2, CD117, C-Met, PTHR2, and HAVCR2 (TIM3).

In some cases, a subject anti-CD24/Siglec10 agent is used in a combination therapy (is co-administered) with any convenient immunomodulatory agent (e.g., an anti-CTLA4 antibody, an anti-PD-1 antibody, an anti-PD-L1 antibody, a CD40 agonist, a 4-1BB modulator (e.g., a 41BB-agonist), and the like). In some cases, a subject anti-CD24/Siglec10 agent is used in a combination therapy (is co-administered) with an anti-CD47/SIRPA agent and any convenient immunomodulatory agent (e.g., an anti-CTLA4 antibody; an anti-PD-1 antibody, an anti-PD-L1 antibody, a CD40 agonist, a 4-1BB modulator (e.g., a 41BB-agonist), and the like). In some cases, a subject anti-CD24/Siglec10 agent is used in a combination therapy (is co-administered) with an inhibitor of BTLA and/or CD160. In some cases, a subject anti-CD24/Siglec10 agent is used in a combination therapy (is co-administered) with an anti-CD47/SIRPA agent and an inhibitor of BTLA and/or CD160. In some cases, a subject anti-CD24/Siglec10 agent is used in a combination therapy (is co-administered) with an inhibitor of TIM3 and/or CEACAM1. In some cases, a subject anti-CD24/Siglec10 agent is used in a combination therapy (is co-administered) with an anti-CD47/SIRPA agent and an inhibitor of TIM3 and/or CEACAM1.

Treatment may also be combined with other active agents, such as antibiotics, cytokines, anti-viral agents, etc. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment.

A “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of an anti-CD24/Siglec10 agent and/or an anti-CD47/SIRPA agent is an amount that is sufficient to palliate, ameliorate, stabilize, reverse, prevent, slow or delay the progression of the disease state (e.g., cancer or chronic infection) by increasing phagocytosis of a target cell (e.g., a target cell). Thus, a therapeutically effective dose of an anti-CD24/Siglec10 agent and/or an anti-CD47/SIRPA agent reduces the binding of (i) CD24 on an target cell, to Siglec10 on a phagocytic cell; and/or (ii) CD47 on an target cell, to SIRPA on a phagocytic cell; at an effective dose for increasing the phagocytosis of the target cell.

In some embodiments, a therapeutically effective dose leads to sustained serum levels of an anti-CD24/Siglec10 agent and/or an anti-CD47/SIRPA agent (e.g., an anti-CD24 antibody, anti-Siglec10 antibody and/or an anti-CD47 antibody) of 40 μg/ml or more (e.g, 50 ug/ml or more, 60 ug/ml or more, 75 ug/ml or more, 100 ug/ml or more, 125 ug/ml or more, or 150 ug/ml or more) for each agent. In some embodiments, a therapeutically effective dose leads to sustained serum levels of an anti-CD24/Siglec10 agent and/or an anti-CD47/SIRPA agent (e.g., an anti-CD24 or Siglec10 antibody and/or an anti-CD47 antibody) that range from 40 μg/ml to 300 ug/ml (e.g, from 40 ug/ml to 250 ug/ml, from 40 ug/ml to 200 ug/ml, from 40 ug/ml to 150 ug/ml, from 40 ug/ml to 100 ug/ml, from 50 ug/ml to 300 ug/ml, from 50 ug/ml to 250 ug/ml, from 50 ug/ml to 200 ug/ml, from 50 ug/ml to 150 ug/ml, from 75 ug/ml to 300 ug/ml, from 75 ug/ml to 250 ug/ml, from 75 ug/ml to 200 ug/ml, from 75 ug/ml to 150 ug/ml, from 100 ug/ml to 300 ug/ml, from 100 ug/ml to 250 ug/ml, or from 100 ug/ml to 200 ug/ml) for each agent. In some embodiments, a therapeutically effective dose for treating solid tumors leads to sustained serum levels of an anti-CD24/Siglec10 agent and/or an anti-CD47/SIRPA agent (e.g., anti-CD24 antibody, anti-Siglec10 antibody and/or an anti-CD47 antibody) of 100 μg/ml or more (e.g., sustained serum levels that range from 100 ug/ml to 200 ug/ml) for each agent. In some embodiments, a therapeutically effective dose for treating non-solid tumors (e.g., acute myeloid leukemia (AML)) leads to sustained serum levels of an anti-CD24/Siglec10 agent and/or an anti-CD47/SIRPA agent (e.g., anti-CD24 antibody, anti-Siglec10 antibody and/or an anti-CD47 antibody) of 50 μg/ml or more (e.g., sustained serum levels of 75 μg/ml or more; or sustained serum levels that range from 50 ug/ml to 150 ug/ml) for each agent.

Accordingly, a single therapeutically effective dose or a series of therapeutically effective doses would be able to achieve and maintain a serum level of an anti-CD24/Siglec10 agent and/or an anti-CD47/SIRPA agent. A therapeutically effective dose of an anti-CD24/Siglec10 agent and/or an anti-CD47/SIRPA agent can depend on the specific agent used, but is usually 8 mg/kg body weight or more (e.g., 8 mg/kg or more, 10 mg/kg or more, 15 mg/kg or more, 20 mg/kg or more, 25 mg/kg or more, 30 mg/kg or more, 35 mg/kg or more, or 40 mg/kg or more) for each agent, or from 10 mg/kg to 40 mg/kg (e.g., from 10 mg/kg to 35 mg/kg, or from 10 mg/kg to 30 mg/kg) for each agent. The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art. For all therapeutically effective doses listed above, when both an anti-CD24/Siglec10 agent and an anti-CD47/SIRPA agent are used, the dose for each agent can be independent from the other agent. As an illustrative example (to illustrate the independence of the doses), a therapeutic dose of the anti-CD24/Siglec10 agent may be from 75 ug/ml to 250 ug/ml while a therapeutic dose of the anti-CD47/SIRPA agent may be from 40 ug/ml to 100 ug/ml.

Dosage and frequency may vary depending on the half-life of the anti-CD24/Siglec10 agent and/or anti-CD47/SIRPA agent in the patient. It will be understood by one of skill in the art that such guidelines will be adjusted for the molecular weight of the active agent, e.g. in the use of antibody fragments, in the use of antibody conjugates, in the use of anti-CD24/Siglec10 agents, in the use of anti-CD47/SIRPA agents, etc. The dosage may also be varied for localized administration, e.g. intranasal, inhalation, etc., or for systemic administration, e.g. i.m., i.p., i.v., and the like.

An anti-CD24/Siglec10 agent and/or an anti-CD47/SIRPA agent can be administered by any suitable means, including topical, oral, parenteral, intrapulmonary, and intranasal. Parenteral infusions include intramuscular, intravenous (bollus or slow drip), intraarterial, intraperitoneal, intrathecal or subcutaneous administration. An anti-CD24/Siglec10 agent and/or an anti-CD47/SIRPA agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants. Localized delivery is particularly contemplated, by such means as delivery via a catheter to one or more arteries, such as the renal artery or a vessel supplying a localized tumor.

As noted above, an anti-CD24/Siglec10 agent and/or an anti-CD47/SIRPA agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An anti-CD24/Siglec10 agent and/or an anti-CD47/SIRPA agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an anti-CD24/Siglec10 agent and/or an anti-CD47/SIRPA agent by non-covalent associations, such as non-covalent bonding or by encapsulation. The nature of the carrier can be either soluble or insoluble for purposes of the invention. Those skilled in the art will know of other suitable carriers for binding anti-CD24/Siglec10 agents and/or anti-CD47/SIRPA agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the anti-CD24/Siglec10 agent and/or an anti-CD47/SIRPA agent through an acceptable chemical linker or chelation carrier. Positron emitting moieties for use in the present invention include ¹⁸F, which can be easily conjugated by a fluorination reaction with the anti-CD24/Siglec10 agent and/or an anti-CD47/SIRPA agent.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the anti-CD24/Siglec10 agents and/or anti-CD47/SIRPA agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

In some cases, a method of inducing phagocytosis of a target cell, treating an individual having cancer, treating an individual having an intracellular pathogen infection (e.g., a chronic infection), and/or reducing the number of inflicted cells (e.g., cancer cells, cells infected with an intracellular pathogen, etc.) in an individual, includes, as described below, predicting whether an individual is resistant or susceptible to treatment with an anti-CD47/SIRPA agent.

### Method of Predicting

As discussed above, in some cases, a target cell (even one that expressed CD47) is relatively resistant to an anti-CD47/SIRPA agent, meaning that the target cell is less susceptible to phagocytosis by a phagocytic cell (e.g., a macrophage), even when the target cell is contacted by a phagocytic cell in the present of an anti-CD47/SIRPA agent. As such, in some cases, an individual can be relatively resistant to treatment with an anti-CD47/SIRPA agent. Expression of CD24 by an inflicted cell can be used to predict whether a target cell (and therefore whether an individual) is resistant to treatment using an anti-CD47/SIRPA agent. In this context, resistance to treatment using an anti-CD47/SIRPA agent refers to treatment in the absence of a subject anti-CD24/Siglec10 agent, because the inventors have discovered that contacting a target cell (e.g., a target cell that is resistant to treatment with an anti-CD47/SIRPA agent) with an anti-CD24/Siglec10 agent can overcome the resistance.

The terms “resistance” and “resistant” (used herein when referring to resistance to an anti-CD47/SIRPA agent) is used herein to refer to target cells that exhibit a decrease in the susceptibility to phagocytosis (in the present of an anti-CD47/SIRPA agent) compared to other cells. For example, while many cancer cells are negative for (or express low levels of) CD24, some cancer cells are positive for CD24. Target cells (e.g., cancer cells) can express CD24 over a range of levels. For example, some target cells express more CD24 than others, but still express less than normal cells. Some target cells express normal levels of CD24. Thus, when the term “resistance” or “resistant” is used, it does not necessarily mean that the cells cannot be phagocytosed, but does mean that the cells are not phagocytosed as efficiently as other cells (e.g., a smaller proportion of cells of a population of the cells can be phagocytosed, e.g., over a given period of time, when compared to other cells).

In some embodiments, a target cell that is resistant to treatment with an anti-CD47/SIRPA agent exhibits a phagocytosis efficiency that is 95% or less (e.g., 90% or less, 85% or less, 80% or less, 75% or less, 70% or less, 65% or less, 60% or less, 55% or less, 50% or less, 45% or less, 50% or less, 45% or less, 40% or less, 35% or less, 30% or less, 25% or less, 20% or less, 15% or less, or 10% or less) of the phagocytosis efficiency exhibited by a control cell (e.g., a control population of cells). Assays to determine phagocytosis efficiency will be known to one of ordinary skill in the art and any convenient assay can be used. As such, an individual can be predicted to be resistant to treatment with an anti-CD47/SIRPA agent when a target cell exhibits an CD24 expression level that is above a particular threshold (which can be determined by comparing the measured expression level to a level measured from a control cell that is susceptible to treatment with an anti-CD47/SIRPA agent.

In some embodiments, a target cell (or an individual) is predicted to be susceptible to an anti-CD47/SIRPA agent when the target cell expresses 95% or less (e.g., 90% or less, 85% or less, 80% or less, 75% or less, 70% or less, 65% or less, 60% or less, 55% or less, 50% or less, 45% or less, 50% or less, 45% or less, 40% or less, 35% or less, 30% or less, 25% or less, 20% or less, 15% or less, or 10% or less) CD24 as expressed by a control cell. In some cases, a target cell (or an individual) is predicted to be resistant to an anti-CD47/SIRPA agent when the target cell expresses 1.1-fold or more (e.g., 1.2-fold or more, 1.3-fold or more, 1.4-fold or more, 1.5-fold or more, 1.6-fold or more, 1.7-fold or more, 1.8-fold or more, 1.9-fold or more, 2-fold or more, 2.1-fold or more, 2.5-fold or more, 3-fold or more, 4-fold or more, 5-fold or more, etc.) CD24 compared to a control cell (e.g., an CD24 negative cell, a cell that expresses low levels of CD24 but is known to be susceptible, and the like) or compared to a background value.

Methods of predicting whether target cells are (or an individual is) resistant or susceptible to treatment with an anti-CD47/SIRPA agent include the step of measuring the expression level of CD24 in a biological sample of the individual to produce a measured test value. The measured test value can then be compared to a control value. In some cases, the value is measured for individual cells (e.g., using flow cytometry).

In some cases, when the measured test value is greater than or equal to the control value, a prediction of resistance is made (and when the measured test value is less than the control value, a prediction of susceptible is made). The control value can be a predetermined value or can be a value that is measured around the same time that the test value is measured. In some cases, the control value is a value of expression which is known to be associated with a phenotype of resistance to an anti-CD47/SIRPA agent. As such, when the measured test value is equal to or greater than this value, a prediction of resistance can be made. Such a control value (one that is known to be associated with a phenotype of resistance to an anti-CD47/SIRPA agent) can be a value measured from an inflicted cell known to exhibit a phenotype of resistance.

In some cases, when the measured test value is greater than the control value, a prediction of resistance is made (and when the measured test value is less than or equal to the control value, a prediction of susceptible is made). The control value can be a predetermined value or can be a value that is measured at or around the same time that the test value is measured. In some cases, the control value is a value representing the background value of the measuring step (e.g., the experiment in which the measurement was performed). For example, in some cases, for a cell to exhibit a phenotype of resistance, the cell only needs to be positive for CD24.

In some cases, when a prediction of resistance is made, the method further includes treating the individual (i.e., contacting the target cell(s)) with an anti-CD24/Siglec10 agent and an anti-CD47/SIRPA agent (e.g., co-administration to the individual, contacting the target cell with a phagocytic cell in vitro in the presence of an anti-CD24/Siglec10 agent and an anti-CD47/SIRPA agent, etc.).

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Measuring may be relative or absolute. For example, “measuring” can be determining whether the expression level is less than or “greater than or equal to” a particular threshold, (the threshold can be pre-determined or can be determined by assaying a control sample). On the other hand, “measuring to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple biological samples from the same individual (e.g., biological samples taken at different points in time from the same individual). This is because the units cancel when calculating a fold-change in the expression level from one biological sample to then next (e.g., biological samples taken at different points in time from the same individual).

The term “measuring” is used herein to include the physical steps of manipulating a biological sample to generate data related to the sample. As will be readily understood by one of ordinary skill in the art, a biological sample must be “obtained” prior to assaying the sample. Thus, the term “measuring” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample. In some cases, the measured expression level of CD24 is normalized (e.g., to an internal experimental control).

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

Measuring the expression level generally entails measuring the expression level of CD24 on or in a cell. In some cases, the methods include measuring the expression level of CD24 on the surface of a cell (e.g., via flow cytometry). In some cases, the methods include measuring the expression level of CD24 in a cell (e.g., via Western Blot, ELISA assay, mass spectrometry, etc).

For measuring protein levels, the amount or level of a polypeptide in the biological sample is determined, e.g., the protein/polypeptide encoded by the biomarker gene. In some cases, the surface protein level is measured. In some cases, the cells are removed from the biological sample (e.g., via centrifugation, via adhering cells to a dish or to plastic, etc.) prior to measuring the expression level. In some cases, the intracellular protein level is measured (e.g., by lysing the cells of the biological sample to measure the level of protein in the cellular contents). In some cases, cells of the biological sample are identified as target cells (e.g., inflicted cells) (e.g., via cell sorting, via microscopic evaluation, via marker analysis, etc.) prior to measuring the expression level of CD24. In some cases, cells of the biological sample are identified as target cells simultaneous with measuring the expression level of CD24 (e.g., via flow cytometry), In some cases, surface levels of CD24 can be measured by extracting or otherwise enriching for or purifying surface proteins, prior to the measuring.

In some instances, the expression level of one or more additional proteins may also be measured, and the level of biomarker expression compared to the level of the one or more additional proteins to provide a normalized value for the biomarker expression level. Any convenient protocol for evaluating protein levels may be employed wherein the level of one or more proteins in the assayed sample is determined.

While a variety of different manners of assaying for protein levels are known to one of ordinary skill in the art and any convenient method may be used, representative methods include but are not limited to antibody-based methods (e.g., flow cytometry, ELISA, Western blotting, proteomic arrays, xMAP™ microsphere technology (e.g., Luminex technology), immunohistochemistry, flow cytometry, and the like); as well as non-antibody-based methods (e.g., mass spectrometry).

When a prediction is made in the subject methods, the methods include a step of providing the prediction. The term “providing a prediction” is not simply a mental step, but instead includes the active step of reporting the prediction either by generating or report, or by orally providing the prediction. In some cases, the prediction is provided as a report. Thus, in some instances, the subject methods may further include a step of generating or outputting a report providing the results of the evaluation of the sample, which report can be provided in the form of a non-transient electronic medium (e.g., an electronic display on a computer monitor, stored in memory, etc.), or in the form of a tangible medium (e.g., a report printed on paper or other tangible medium). Any form of report may be provided, e.g. as known in the art or as described in greater detail below.

In some embodiments, a report is generated. A “report,” as described herein, is an electronic or tangible document which includes report elements that provide information of interest relating to the assessment of a subject and its results. In some embodiments, a subject report includes the measured test value that represents the measured expression level of CD24 (e.g., the normalized measured expression level). In some embodiments, a subject report includes an artisan's assessment, e.g. a prediction of resistance or susceptibility, a treatment recommendation, a prescription, etc. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a protein level determination; 6) other features.

In some embodiments, a prediction is provided by generating a written report. Thus, the subject methods may include a step of generating or outputting a report, which report can be provided in the form of an electronic medium (e.g., an electronic display on a computer monitor), or in the form of a tangible medium (e.g., a report printed on paper or other tangible medium). Any form of report may be provided.

The report may include a sample data section, which may provide information about the biological sample analyzed in the monitoring assessment, such as the source of biological sample obtained from the patient (e.g. Tumor, blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu). The report may include a results section. For example, the report may include a section reporting the results of a marker expression level determination assay, or a prediction of resistance or susceptibility.

### Kits

Also provided are kits for use in the methods. The subject kits can include an anti-CD24/Siglec10 agent and/or an anti-CD47/SIRPA agent and/or an antibody specific for a target cell. In some embodiments, an anti-CD24/Siglec10 agent is provided in a dosage form (e.g., a therapeutically effective dosage form. In the context of a kit, an anti-CD24/Siglec10 agent can be provided in liquid or sold form in any convenient packaging (e.g., stick pack, dose pack, etc.). The agents of a kit can be present in the same or separate containers. The agents may also be present in the same container. In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### The CD24-Siglec10 Signaling Axis is a Target for Cancer Immunotherapy by Macrophages

Three signaling axes have been previously demonstrated to inhibit phagocytosis: CD47-SIRPα, MHC I-LILRB1, and PD1-PDL1. Despite efforts to block these axes to promote macrophage phagocytosis, there is still resistance to phagocytosis. Even in the presence of monoclonal antibodies antagonizing CD47, some cancer lines exhibit only modest phagocytosis, some cancers do not respond at all to anti-CD47 treatment, and among cancers which do respond, not all cancer cells are phagocytosed (Barkal, A. A. et al. Engagement of MHC class I by the inhibitory receptor LILRB1 suppresses macrophages and is a target of cancer immunotherapy. Nature Immunology 19, 76-84 (2017)). We hypothesized that this continued resistance to phagocytosis is due to the presence of additional “don't eat me” signals expressed by cancer cells.

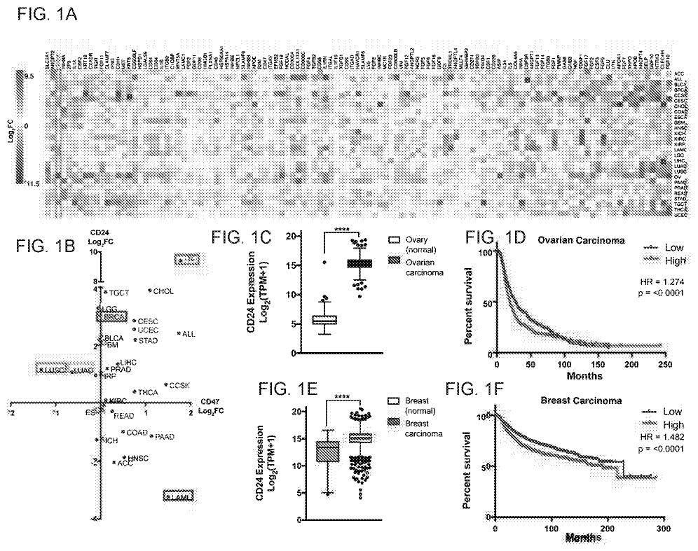

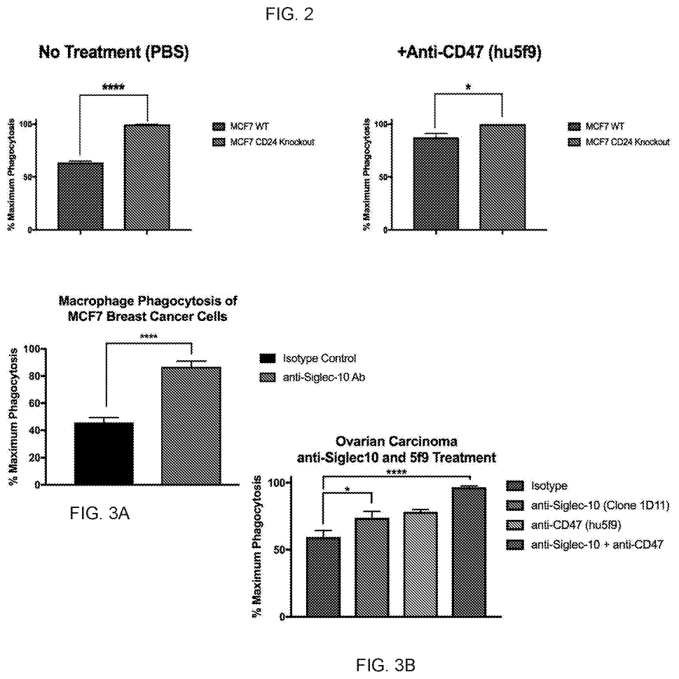

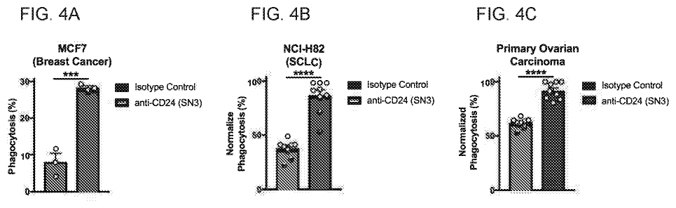

A novel “don't eat me” signaling axis was uncovered that exists between CD24 expressed by cancer cells and the inhibitory macrophage receptor Siglec10. CD24 engages the macrophage inhibitory receptor Siglec10 in order to inhibit phagocytosis. CD24 expression is upregulated on cancer cells versus tissue-matched normal cells (FIG. 1A-C,E) and is an adverse prognostic indicator in multiple cancers (FIG. 1D,F). By antagonizing CD24-Siglec10 signaling we promote macrophage-mediated clearance of cancer cells. CD24^{-/-} MCF7 cells are more susceptible to phagocytosis by human macrophages in vitro (FIG. 2). Additionally, we show that monoclonal antibodies targeting Siglec10 (FIG. 3) and CD24 (FIG. 4) promote the phagocytosis of breast cancer cells, small cell lung cancer cells, and primary ovarian carcinoma.

Blocking CD24-Siglec10 signaling provides clinical opportunities to augment macrophage phagocytosis of cancer cells and thus tumor burden. CD24 expression also provides a biomarker for response to existing macrophage-targeting immune therapies, and allows selection of appropriate therapy based on CD24 expression.

### Materials and Methods

In vitro phagocytosis assays. Phagocytosis assays were performed with primary human monocyte-derived macrophages (MDMs) as described previously. Briefly, fluorescently labeled cancer cells were incubated with MDMs at a ratio of 2:1 in IMDM without serum at 37° C. for 2 hours. Blocking antibodies or isotype controls were used at a concentration of 10 micrograms per mL per reaction well. Phagocytosis reactions were quenched by the addition of ice cold PBS and reactions were stained with conjugated anti-CD11b antibody to label macrophages. Reactions were analyzed using flow cytometry to quantify phagocytosis events, as defined by CD11b+ macrophages also positive for the cancer cell fluorescent label (RFP or GFP).

### Results

We found that a number of cancers analyzed (13/27) upregulate both CD24 and CD47 mRNA expression (FIG. 2B). Notably, CD24 is dramatically upregulated in ovarian carcinoma (OV) (FIGS. 2B, C) and substantially upregulated in breast carcinoma (BRCA) (FIGS. 2B, E). It is also of interest that both lung squamous cell carcinoma and lung adenocarcinoma (LVAD) upregulate CD24 expression and not CD47 expression. Interestingly, CD24 is downregulated in Acute Myeloid Leukemia, which may be due to cis inhibitory interactions between myeloid CD24 and Siglec10 (FIG. 2B). To determine whether CD24 RNA mRNA expression levels are a prognostic factor in human tumors, we analyzed gene-expression data from human solid tumors (Km-Plotter). In a univariate analysis, patients were stratified into “CD24 high” and “CD24 low” groups based on an optimum threshold. We found that CD24 mRNA expression levels were correlated with decreased probability of overall survival in both breast carcinoma (FIG. 2D) and ovarian carcinoma (FIG. 2F). These results show the utility for CD24 mRNA expression levels as a prognostic factor in certain solid tumors.

To validate our finding that CD24 can directly inhibit phagocytosis by macrophages, we engineered MCF7 breast cancer cells lacking CD24 using CRISPR/Cas9 to lead to a frame-shift mediated knockout within the first exon of the CD24 gene locus. Co-culture of these lines with donor-derived human macrophages revealed that even in the absence of any additional pro-phagocytic stimuli, cancer cells lacking CD24 were significantly more susceptible to phagocytosis (FIG. 2A). Similarly, in the presence of CD47-blockade through a monoclonal antibody (5F9-G4), CD24−/− MCF7 cells were more susceptible to phagocytosis than MCF7 WT cells also treated with CD47 blockade (FIG. 2B).

We next tested the ability for monoclonal antibodies against Siglec10 to promote phagocytosis by preventing the interaction between the macrophage inhibitory receptor Siglec10 and CD24. We observed a dramatic increase in phagocytosis of MCF7 cells in the presence of Siglec10 antibody. Furthermore, primary ovarian carcinoma cells are more readily phagocytosed by macrophages in the presence of Siglec10 monoclonal antibodies (FIG. 3A). This effect was enhanced by the addition of CD47-blocking antibodies (5F9-G4) (FIG. 3B). To extend these results, we next evaluated the ability of monoclonal antibodies against CD24 to promote phagocytosis in vitro. Consistent with our expectations, blockade of CD24 significantly increased phagocytosis for all three cell lines tested: the MCF7 breast cancer cells, NCI-H82 small cell lung cancer cells, and primary ovarian carcinoma (FIG. 4A-C).

### Example 2

### CD24 Signaling Through Macrophage Siglec-10 is a New Target for Cancer Immunotherapy

Ovarian cancer and triple-negative breast cancer (TNBC) are among the most lethal diseases affecting women, with few targeted therapies and high rates of metastasis. Here we show that CD24 can be the dominant innate immune checkpoint in ovarian cancer and breast cancer, and is a novel, promising target for cancer immunotherapy. Cancer cells are capable of evading attack and clearance by macrophages through the overexpression of anti-phagocytic surface proteins, called “don't eat me” signals. Known “don't eat me” signals CD47, programmed cell death ligand 1 (PD-L1), and the beta-2 microglobulin subunit of the major histocompatibility class I complex (B2M)₅ often represent the appropriation of mechanisms for self-nonself discrimination as a means of immune escape. Monoclonal antibodies selected for their ability to antagonize the interaction of these “don't eat me” signals with their macrophage-expressed receptors have demonstrated therapeutic potential in a variety of cancers. However, variability in the magnitude and durability of the response to these agents has suggested the presence of additional, as yet unknown “don't eat me” signals. Here we demonstrate a novel role for tumor expressed CD24 in promoting immune evasion by cancer cells through its interaction with the inhibitory receptor, Sialic Acid Binding Ig Like Lectin 10 (Siglec-10), expressed by tumor associated macrophages (TAMs). We observed that many tumors overexpress CD24 and that TAMs express high levels of Siglec-10. Both genetic ablation of CD24 or Siglec-10 and monoclonal antibody blockade of the CD24-Siglec-10 interaction robustly augment the phagocytosis of all CD24-expressing human tumors tested, up to 13-fold that of baseline, in many cases outperforming CD47 blockade. Furthermore, genetic ablation of CD24 in a human xenograft tumor model of breast cancer resulted in a macrophage-dependent reduction of tumor growth and extension of survival. These data highlight CD24 as a dominant anti-phagocytic signal, and critical regulator for innate immune activity in several cancers, especially in ovarian cancer and breast cancer. These findings represent a significant advance in our understanding of these diseases and demonstrate the potential for CD24-blockade as an effective therapeutic strategy.

CD24, also known as Heat Stable Antigen (HSA) or Small Cell Lung Carcinoma Cluster 4 Antigen, is a heavily glycosylated GPI-anchored surface protein known to interact with Siglec-10 expressed on innate immune cells in order to dampen damaging inflammatory responses to infection, sepsis, liver damage, and chronic graft versus host disease. The binding of sialylated CD24 to Siglec-10 on immune cells, including macrophages, elicits an inhibitory signaling cascade mediated by SHP-1 and/or SHP-2 phosphatases associated with the two immunoreceptor tyrosine-based inhibition motifs on the cytoplasmic tail of Siglec-10, thereby blocking TLR-mediated inflammation and the cytoskeletal rearrangement required for cellular engulfment by macrophages. Studies have shown that CD24 is expressed by several solid tumors, however a role for CD24 in modulating tumor immune responses has not yet been shown. We thus sought to investigate whether CD24-mediated inhibition of the innate immune system could be harnessed by cancer cells as a mechanism to avoid detection and clearance by immune cells expressing Siglec-10.

To assess the role of CD24-Siglec-10 signaling in regulating the macrophage-mediated immune response to cancer, we examined the expression of CD24 and Siglec-10 expression in various tumors and associated immune cells.

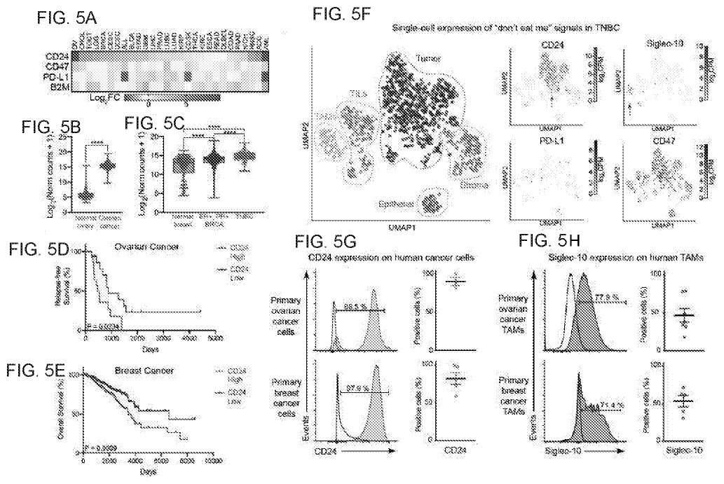

Publicly available RNA-sequencing data from TCGA and TARGET demonstrated high expression of CD24 in nearly all tumors analyzed (FIG. 13a. Tumor study abbreviations, Table 1), as well as broad upregulation of tumor CD24 expression in several tumors as compared to the known innate immune checkpoints, CD47, PD-L1, and B2M (FIG. 5a). The greatest CD24 upregulation was observed in ovarian cancer (OV), over 9 log-fold; and, CD24 expression in TNBC was significantly higher than that in either normal breast, or ER+PR+ breast cancers (FIG. 5b,c). Stratification of TOGA patients by high or low CD24 expression relative to median CD24 expression revealed increased relapse free survival for OV patients with lower CD24 expression, and an overall survival advantage for patients with lower CD24 expression in breast cancer (FIG. 5d,e).

Because these tumor studies assayed mixtures of many cell types, with varying frequencies of cancer cells versus non tumorigenic cells, we next tested CD24 and Siglec-10 expression on a cellular level within the tumor by leveraging single-cell RNA-sequencing data from six primary samples of TNBC (NCBI SRA: PRJNA485423). Cells were clustered using a set of previously defined marker genes revealing distinct cell populations within the tumors including TAMs, tumor infiltrating lymphocytes (TILs), tumor epithelial cells, stomal cells, and a distinct epithelial cell population, with cells from patients distributed among each cluster (FIG. 5f, FIG. 13b,c). TNBC cancer cells exhibited robust expression of CD24 across each patient (FIG. 5f), while all other cell clusters exhibited weak CD24 expression, thus illustrating the potential for CD24 as a tumor-associated cellular marker. CD47 was found to be expressed by all cell types (FIG. 5f). A substantial fraction of TAMs were found to express Siglec-10 (FIG. 5f) indicating the possibility for CD24-Siglec-10 interactions in TNBC (FIG. 50. The expression of PD-L1 (CD274) was substantially lower than that of CD24 in all patients (FIG. 13d), suggesting that patients with TNBC may be poor candidates for PD-L1-mediated checkpoint blockade therapies.

FACS analyses of primary human tumor samples revealed robust CD24 protein expression by EpCAM+ breast cancer cells as well as by ovarian cancer cells from malignant ascites (FIG. 5g, FIG. 14a). TAMs from both ovarian cancer ascites and primary breast cancer expressed Siglec-10 (FIG. 5h). In contrast, human macrophages collected from non-cancerous ascites fluid expressed substantially lower levels of Siglec-10 than observed in TAMs (FIG. 14b). Analysis of PBMC subsets revealed low expression of Siglec-10 and CD24 in T cells, NK cells, and monocytes, whereas B cells were found to express modest levels of both Siglec-10 and high levels of CD24 (FIG. 14c,d).

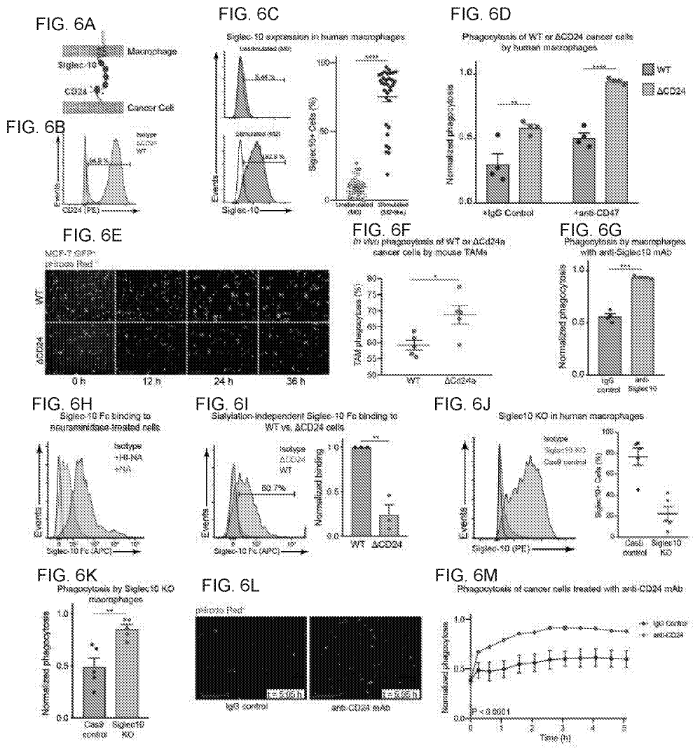

Given that CD24 is expressed by human cancer cells and its receptor Siglec-10 is expressed by TAMs, we tested whether CD24 can inhibit macrophage-mediated clearance of cancer via Siglec-10 (FIG. 6a). In order to investigate this role for CD24-Siglec-10 signaling in regulating the anti-tumor immune response, we tested the MCF-7 human breast cancer cell line which was found at baseline to be >90% CD24+(WT). We engineered a polyclonal subline of MCF-7 cells deficient in CD24 (ΔCD24) through stable genetic knockout (FIG. 6b). Although unstimulated (M0) human donor-derived macrophages were found to express very low levels of Siglec-10 by FACS, the addition of two inhibitory cytokines, TGFβ-1 and IL-10, induced robust expression of Siglec-10, indicating that Siglec-10 expression may be regulated by TAM-specific gene expression programs (FIG. 6c). Due to the inhibitory stimulation, these TGFβ-1, IL-10⁻ stimulated macrophages were found to be less phagocytic than unstimulated macrophages at baseline (FIG. 14e). Notably, stimulation with the classic M2-polarizing cytokine, IL-4, was also sufficient to induce robust Siglec10 expression in most patients, comparable to that induced by TGFβ-1 and IL-10 (FIG. 14f).

Co-culture of either WT or ΔCD24 cells with TGFβ-1, IL-10-stimulated (M2-like) macrophages expressing Siglec-10 revealed that CD24 deletion alone was sufficient to potentiate spontaneous phagocytosis. ΔCD24 cells were also significantly more sensitive to anti-CD47 blockade (Clone 5F9-G4), than WT cells, suggesting the cooperativity of combinatorial blockade of CD24 and CD47 (FIG. 6d). To measure phagocytic clearance by automated live cell microscopy, GFP+WT and ΔCD24 cells were labeled with the pH-sensitive dye, pHrodo red, and co-cultured with primary human macrophages. We observed that ΔCD24 cells were much more readily engulfed into the low-pH phagolysosome as indicated by an increase in red signal and subsequent loss of GFP signal as compared to WT cells, over 36 hours (FIG. 6e).

To determine whether the mouse homolog of human CD24, Cd24a, could similarly confer protection against phagocytic clearance of cancer cells, we generated a subline of the mouse epithelial ovarian cancer line, ID8, lacking Cd24a (ID8ΔCd24a). In order to recapitulate the microenvironment of malignant ovarian cancer ascites, WT or ΔCd24a cells expressing GFP were injected intraperitoneally into mice of the NOD.Cg-Prkdc_{SCID}II2rgₜₘ₁wⱼₗ/SzJ (NSG) background which produce functional cells of the myeloid lineage, but lack B, T, and NK cells, in order to observe the myeloid-specific effect of Cd24a deletion. After one week, peritoneal cells were harvested by lavage and phagocytosis was measured by FACS as defined by the number of CD11b⁺F4/80⁺ macrophages which were also GFP⁺. Loss of Cd24a was sufficient to significantly promote phagocytic engulfment by mouse peritoneal macrophages as compared to WT cells, indicating that the role for CD24 in protecting cells from phagocytic clearance is conserved across both humans and mice (FIG. 6f).

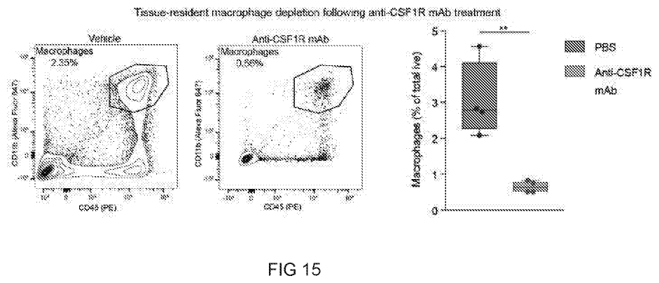

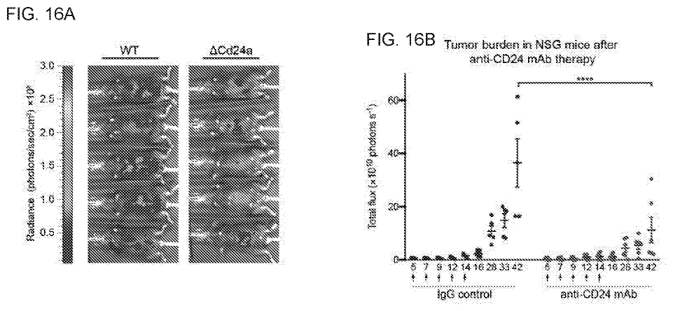

We next sought to investigate the functional role for Siglec10 in mediating the anti-phagocytic signal conferred by CD24. Direct blockade of Siglec10 on donor-derived macrophages through monoclonal antibodies significantly augmented the phagocytosis of parental MCF-7 cells in the absence of additional stimulation, confirming a functional role for Siglec10 in inhibiting phagocytosis (FIG. 6g). Siglec10 has been reported to interact with the highly sialylated form of CD24. Accordingly, we observed that recombinant Siglec10-Fc binding to parental MCF-7 cells was significantly reduced, although not completely abrogated upon surface desialylation through neuraminidase treatment (FIG. 6h, FIG. 15b). This suggests that Siglec10 has the capacity to recognize both protein and sialic acid ligands, and thus likely has varied ligands extending beyond CD24. Indeed, we observed that CD24 deletion alone is insufficient to completely abrogate Siglec-10-Fc binding in the presence of surface sialylation (FIG. 15a,b). However, following removal of cell surface sialylation through neuraminidase treatment, Siglec10-Fc binding was nearly abolished by CD24 deletion, suggesting that CD24 is a primary protein ligand for Siglec10, independent of sialylation (FIG. 6i, FIG. 15b). We found that desialylation through neuraminidase treatment did not reduce the enhancement of phagocytosis observed with CD24 deletion, indicating that CD24 sialylation is not required to inhibit phagocytosis (FIG. 15c). Neither recombinant Siglec5-Fc nor Siglec9-Fc were found to bind CD24+ MCF-7 cells, although both were expressed at high levels by donor-derived macrophages, further defining the specific interaction between Siglec-10 and CD24 (FIG. 15d-g).

In order to further investigate the impact of Siglec10 expression on phagocytosis, we knocked out the SIGLEC10 gene in donor-derived macrophages using CRISPR/Cas9 ribonucleoproteins. We observed a dramatic reduction in population-wide Siglec10 expression 72 h following electroporation with a single guide RNA (sgRNA) targeting the SIGLEC10 locus, relative to its expression in cells treated with Cas9 alone (Cas9 control) (FIG. 6j). Siglec10 KO macrophages demonstrated significantly greater phagocytic ability than donor-matched Cas9 control macrophages, thereby demonstrating that the elimination of surface Siglec10 was sufficient to potentiate the phagocytosis of CD24+ cells in vitro (FIG. 6k).

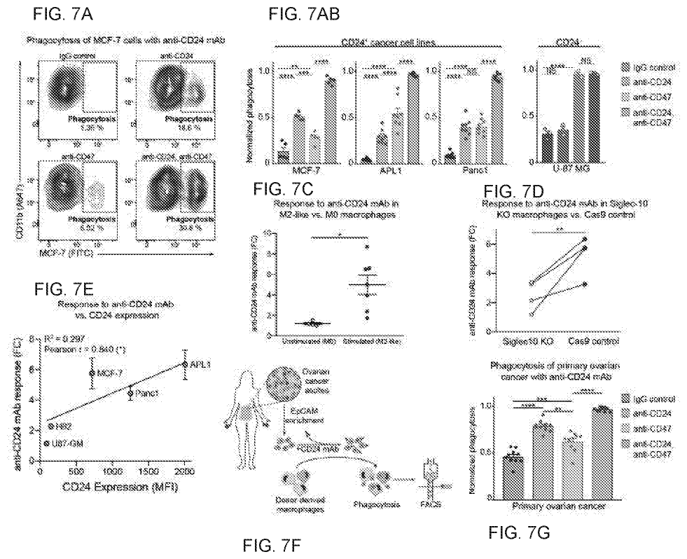

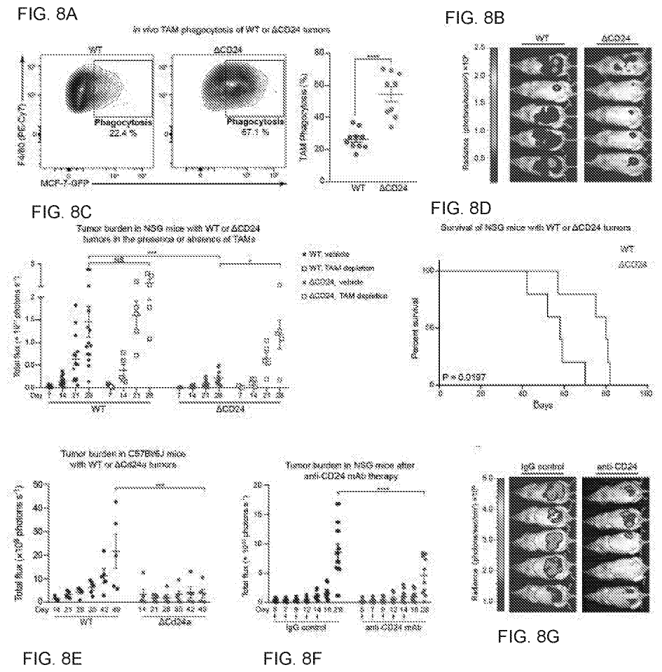

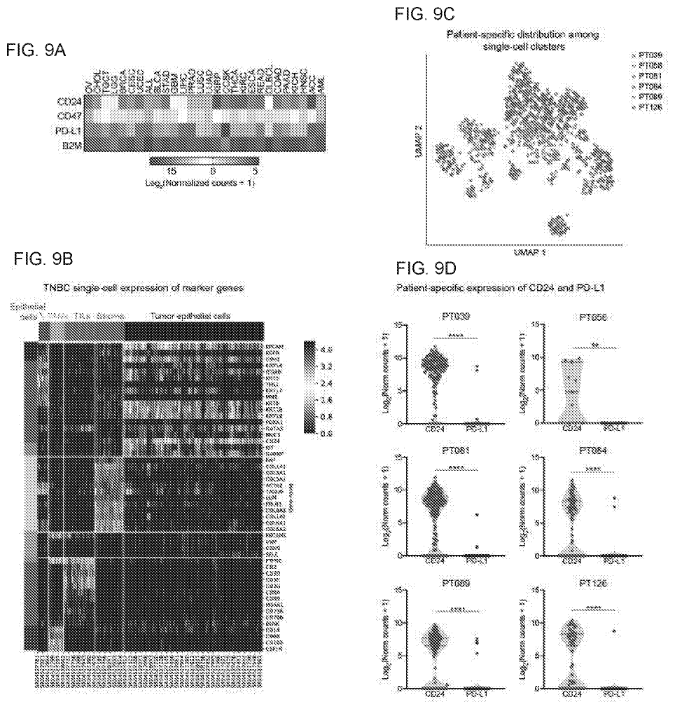

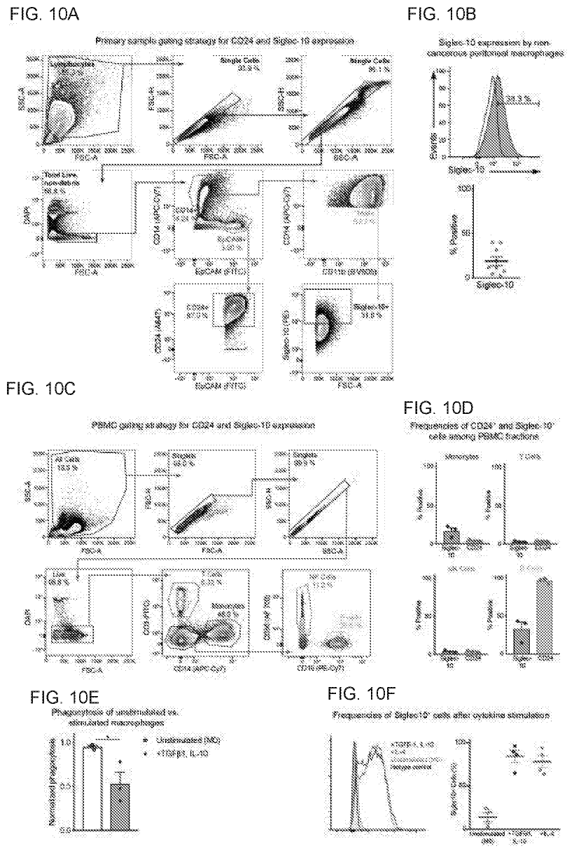

To investigate the human therapeutic potential of these findings, we examined whether direct monoclonal antibody (mAb) blockade of CD24 could enhance the phagocytosis of CD24⁺ cancers by disrupting CD24-Siglec-10 signaling. Automated live-cell microscopy revealed that MCF-7-pHRodo-Red+ cells treated with a CD24 blocking mAb (clone SN3) were much more readily engulfed by macrophages, as demonstrated by an increase in red signal over time as compared to cells treated with an IgG control (FIG. 6l,m). In order to determine the extent of this phenomenon, we measured the phagocytic clearance of various cancer types upon the addition of CD24 mAb by FACS (Extended Data FIG. 8, 5a). This revealed a robust enhancement of phagocytosis of MCF-7 cells treated with CD24 mAb as compared to cells treated with an IgG control, greater than the effect observed with CD47 blockade (FIG. 7a). The response to CD24 mAb was found to be dose-dependent and saturable (FIG. 13b).

To extend those results, we applied the CD24 mAb to a panel of human cancer cell lines found to express CD24. CD24 blockade augmented the phagocytic clearance of all CD24-expressing cancers tested, including breast cancer (MCF-7), pancreatic adenocarcinoma (Panc1), pancreatic neuroendocrine tumor (APL1), and small cell lung cancer (NCI-H82) (FIG. 7b, FIG. 13c). Dual treatment of cancers with CD24 and CD47 blocking antibodies revealed an increased induction of phagocytosis to nearly 30-fold that of baseline in some cancers. The CD24 mAb had no effect on the phagocytosis of the CD24 low expressing U-87 MG glioblastoma cell line (FIG. 7b). Although CD47 genetic deletion did not alter the phagocytic susceptibility of MCF7 cells on its own, upon treatment with anti-CD24 mAb, CD47 KO cells were much more readily engulfed than WT counterparts (FIG. 13d). Dual treatment of Panc1 pancreatic adenocarcinoma cells with anti-CD24 mAb and cetuximab, an opsonizing anti-EGFR mAb, significantly enhanced phagocytosis to levels above either treatment alone, demonstrating the potential for synergy between anti-CD24 mAb and anti-solid tumor monoclonal antibodies (FIG. 13e). An isotype-matched antibody against the surface marker EpCAM, expressed highly by MCF-7 cells, led to a modest increase in phagocytosis as compared to treatment with anti-CD24 mAb, indicating that the vast majority of the observed enhancement upon the addition of anti-CD24 mAb is due to the disruption of CD24 signaling and not due to non-specific activation from Fc-mediated opsonization of cancer cells (FIG. 13f).

All stimulated, Siglec-10-expressing donor-derived macrophages responded to CD24 blockade (FIG. 7c), and response magnitude trended towards a correlation with Siglec-10 expression among stimulated macrophages (FIG. 13g). Notably, unstimulated, M0, macrophages exhibiting low levels of Siglec-10 demonstrated reduced response to CD24 mAb (FIG. 7c). There were no detectable differences in response of either M0 or M2-like, Siglec10+ macrophages to Fc-mediated opsonization by anti-EpCAM antibody (FIG. 13h). Furthermore, genetic deletion of Siglec10 among stimulated macrophages led to dramatically reduced response to anti-CD24 blockade, indicating that anti-CD24 mAb specifically disrupts CD24− Siglec10 signaling (FIG. 7d). The relative expression levels of CD24 were strongly correlated with response to CD24 blockade as well as with innate baseline phagocytosis levels of CD24₊ cell lines, indicating tissue-specific expression of CD24 as the dominant “don't eat me” signal, and highlighting the potential value for CD24 expression as a predictor of the innate anti-tumor immune response (FIG. 7d, FIG. 13i).

We evaluated the ability of the CD24 mAb to promote the macrophage-mediated clearance of primary human tumors. Samples were collected from the malignant ascites fluid from patients with metastatic ovarian cancer and EpCAM⁺ cancer cells were enriched, fluorescently labeled, and co-cultured with donor-derived macrophages in order to measure phagocytosis (FIG. 7f). Upon applying the CD24 mAb to the ovarian cancer cells, we observed a substantial induction of phagocytosis. In these cases, CD24 mAb yielded a significantly greater induction of phagocytosis as compared to CD47 blockade, and dual treatment with both CD24 blockade and CD47 blockade resulted in at least an additive effect (FIG. 7g). Furthermore, CD24 mAb treatment of primary human TNBC cells promoted clearance by macrophages, while in these cases CD47 blockade had no measured effect on phagocytosis (FIG. 13j). Thus, CD24 mAb has therapeutic potential for the treatment of metastatic cancer cells as well as tumors demonstrating resistance to CD47 blockade.

To investigate whether the protection against phagocytosis conferred by CD24 could be recapitulated in vivo, GFP-luciferase tagged MCF-7 WT or MCF-7ΔCD24 cells were engrafted orthotopically in the mammary fat pad of NSG mice. Three weeks post-engraftment, the resulting tumors were dissociated and in vivo phagocytosis by infiltrating TAMs was assessed by measuring the percentage of TAMs which were also GFP⁺ (FIG. 8a, FIG. 14a). Tumors lacking CD24 (ΔCD24) exhibited augmented levels of TAM phagocytosis as compared to WT counterparts indicating that CD24 is capable of protecting cancer cells from attack by macrophages in vivo (FIG. 8b). Furthermore, we found that TAMs infiltrating the CD24− deficient tumors possessed a more inflammatory phenotype, as indicated by significantly higher CD80 expression (FIG. 14b).

The growth of the GFP-luciferase-expressing WT and ΔCD24 tumors was quantified using bioluminescence imaging and revealed a robust reduction of tumor growth of ΔCD24 tumors as compared to the WT counterparts (FIG. 8b,c). The sub-lines assessed above had no measurable cell-autonomous differences in proliferation in vitro (FIG. 13c). Notably, after 35 days of growth, the polyclonal ΔCD24 tumors had become largely CD24+, consistent with the selection against CD24⁻ cells by TAMs and the emergence of subclones of CD24+ cells that did not have biallelic CD24 deletion (FIG. 14d). TAM depletion did not significantly alter the tumor burden of WT tumors, while loss of TAMs largely abrogated the reduction of tumor growth observed in ΔCD24 tumors indicating that increased TAM-mediated clearance of ΔCD24 cells was responsible for the observed diminished tumor burden (FIG. 8c, FIG. 15, see Methods for TAM depletion protocol). This growth difference due to enhanced phagocytic clearance resulted in a significant survival advantage for mice engrafted with ΔCD24 tumors (FIG. 8d).

In order to determine whether the protection against phagocytosis and tumor growth conferred by CD24 could be recapitulated in a syngeneic, fully immunocompetent mouse model of tumor growth, luciferase+ID8 WT or ID8ΔCd24a ovarian cancer cells were engrafted intraperitoneally into C57Bl/6J mice. Tumor growth was measured over time by bioluminescence imaging and revealed that loss of Cd24a was sufficient to dramatically reduce tumor growth over several weeks (FIG. 8e, FIG. 16a). To demonstrate that this enhancement of anti-tumor immunity could be modulated by therapeutic blockade of CD24, NSG mice with established GFP-luciferase tagged MCF-7 WT (CD24+) tumors were treated with anti-CD24 monoclonal antibody for 2 weeks. Anti-CD24 monotherapy resulted in a significant reduction of tumor growth compared to IgG control, as evaluated by bioluminescence imaging (FIG. 8f, g, FIG. 16b). These data indicate the therapeutic potential for anti-CD24 antibodies in inhibiting growth of human solid tumors.

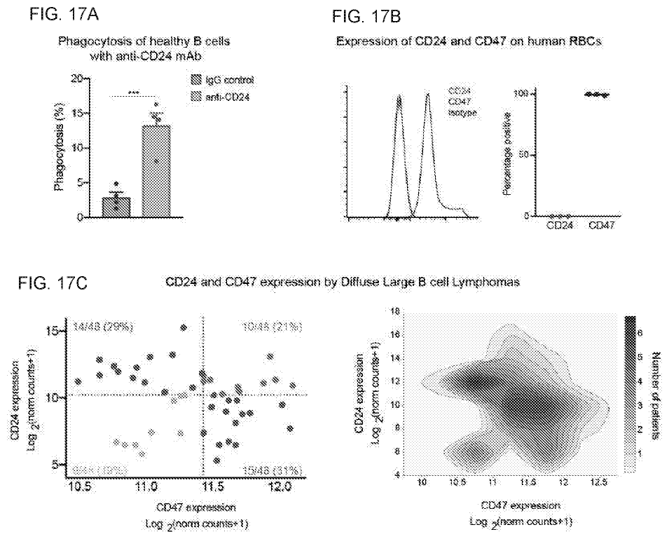

In order to further evaluate blockade of CD24 as a cancer immunotherapeutic strategy, we sought to localize potential off-target effects of anti-CD24 mAb. Phagocytic clearance of healthy B cells was observed upon the addition of anti-CD24 mAb indicating the potential for B cell depletion upon treatment (FIG. 17a). We found that unlike anti-CD47 mAbs, the anti-CD24 mAb demonstrate no detectable binding to human red blood cells (RBCs), indicating that anemia induced by phagocytic clearance of RBCs is unlikely to be observed in humans (FIG. 17b).

Here we show that CD24 is a potent anti-phagocytic, “don't eat me,” signal capable of directly protecting cancer cells from attack by Siglec-10-expressing macrophages. Monoclonal antibody blockade of CD24-Siglec-10 signaling robustly enhances the clearance of CD24⁺ tumors, and has been found to be the dominant anti-phagocytic “don't eat me” signal in the ovarian cancers and breast cancers tested. These findings indicate promise for CD24 blockade in the treatment of CD24⁺ tumors as immunotherapy. Both ovarian cancer and breast cancer have demonstrated weaker responses to anti-PDL-1/PD-1 T cell mediated immunotherapies than those observed in melanoma and non-small cell lung cancer₃₅, which may be attributed to their comparatively lower expression levels of PD-L1, suggesting that an alternate strategy may be required to achieve wide-ranging responses among these tumor types.

Macrophages are often the most plentiful infiltrating immune cells in several cancers, and thus represent potential for targeting by cancer immunotherapy to facilitate direct tumor clearance. Augmenting in situ tumor phagocytosis with these macrophage checkpoint blockade antibodies may lead to in vivo enhancement of adaptive immunity within the tumor through presentation of phagocytosed tumor antigens to T cells. It is notable that the “don't eat me” signals CD47, PD-L1, B2M, and now CD24, each involve ITIM-based macrophage signaling, which may indicate a conserved mechanism that leads to immunoselection of the subset of macrophage-resistant cancer cells, resulting in tumors that by nature avoid macrophage surveillance and clearance.

It is interesting to note the potential for other ITIM-containing Siglecs to modulate phagocytic clearance of cancer cells such as Siglec-9 and Siglec-5 which are widely expressed by TAMs and have been shown to modulate innate immunity through the engagement of protein ligands in cancer and infection. Although we were unable to find evidence of interaction between CD24 and Siglec-5 or Siglec-9, it remains possible that alternative glycoproteoforms of CD24 may create ligands for other Siglecs.

Our findings also indicate that CD24 expression may provide immediate predictive value on responsiveness to existing immunotherapies insofar as high CD24 expression may inhibit response to therapies reliant on macrophage function. As such, expression of CD24 and CD47 was found to be inversely related among Diffuse Large B cell Lymphoma patients (FIG. 17c). The percentage of patients with CD24 over-expression compares well with the response rates observed with anti-CD47 rituximab combination therapy (˜50% ORR, 75% CR), opening the possibility that particular tumors might respond differentially to treatment with anti-CD24 and/or anti-CD47 mAbs.

Collectively, this work defines CD24-Siglec-10 as a novel innate immune checkpoint critical for mediating anti-tumor immunity and provides evidence for the therapeutic potential of CD24 blockade in cancers that express high levels of CD24, with particular promise for the treatment of ovarian cancer and breast cancer.

TABLE 1

| | | | Pattern | Healthy |
|---|---|---|---|---|
| TCGA Study | | | tumors | samples |
| Abbreviation | TCGA or TARGET Study Name | GTEX Study Name(s) | analyzed (n) | analyzed (n) |
| OV | Ovarian serous cystadenocarcinoma | Ovary | 419 | 88 |
| CHOL | Cholangiocarcinoma | N/A (only TCGA normal) | 36 | 9 |
| TGCT | Testicular germ cell tumors | Testis | 148 | 165 |
| LGG | Lower grade glioma | Brain - cortex | 509 | 105 |
| BRCA | Breast invasive carcinoma | Breast | 1092 | 292 |
| CESC | Cervical squamous cell and endocervical adenocarcin [symbol] | Endocervix, Ectocervix | 304 | 13 |
| UCEC | Uterine corpus endometrial carcinoma | N/A (only TCGA normal) | 180 | 23 |
| ALL | Acute lymphoblastic leukemia (peripheral blood) | Whole blood | 37 | 337 |
| BLCA | Bladder urothelial carcinoma | Bladder | 407 | 28 |
| STAD | Stomach adenocarcinoma | Stomach | 414 | 211 |
| GBM | Glioblastoma multiforme | Brain - cortex | 153 | 110 |
| LIHC | Liver hepatocellular carcinoma | Liver | 369 | 160 |
| PRAD | Prostate adenocarcinoma | Prostate | 495 | 152 |
| LUSC | Lung squamous cell carcinoma | Lung | 498 | 50 |
| LUAD | Lung adenocarcinoma | Lung | 513 | 59 |
| KIRP | Kidney renal papillary cell carcinoma | Kidney | 288 | 32 |
| CCSK | Clear cell sarcoma of the kidney (TARGET) | Kidney | 13 | 28 |
| THCA | Thyroid carcinoma | Thyroid | 504 | 338 |
| KIRC | Kidney renal clear cell carcinoma (TCGA) | Kidney | 530 | 72 |
| ESCA | Esophagal carcinoma | Esophagus | 181 | 13 |
| READ | Rectum adenocarcinoma | N/A (only TCGA normal) | 92 | 11 |
| DLBCL | Diffuse large B cell lymphoma | Whole blood | 48 | 337 |
| COAD | Colon adenocarcinoma | Colon - sigmoid, Colon - transver [symbol] | 288 | 41 |
| PAAD | Pancreatic adenocarcinoma | Pancreas | 178 | 171 |
| KICH | Kidney chromophobe | Kidney | 66 | 25 |
| HNSC | Head and neck squamous cell carcinoma | N/A (Only TCGA normal) | 518 | 44 |
| ACC | Adrenocortical carcinoma | Adrenal gland | 77 | 128 |
| AML | Acute Myeloid Leukemia | Whole blood | 200 | 337 |

| Antibody | Clone | Use | Supplier |
|---|---|---|---|
| Anti-human CD24 | SN3 (conjugated) | FACS | Novus Bio, Thermo |
| | | | Fisher Scientific |
| | SN3 (unconjugated) | Treatment | Novus Bio |
| Anti-human Siglec- 10 | 5G6 (conjugated) | FACS | BioLegend |
| | 5G6 (unconjugated) | Treatment | BioLegend |
| Mouse IgG1 isotype control | MOPC-21 (conjugated) | FACS | BioLegend |
| | MOPC-21 (unconjugated) | Treatment | BioXCell |
| Anti-human CD47 | 86H12 | FACS | BioLegend |
| | 5F9-G4 | Treatment | In house |
| Anti-human Fc | HP6017 | FACS | BioLegend |
| Anti-human EGFR | Cetuximab | Treatment | Bristoll-Myers-Squibb |
| Human IgG4 Isotype Control | ET904 | Treatment | Eureka Therapeutics |
| Human IgG1 Isotype Control | N/A (Cat #: BE0297) | Treatment/FACS | BioXCell |
| Anti-human CD14 | M5E2 | FACS | BioLegend |
| Anti-human CD45 | HI30 | FACS | BioLegend |
| Anti-human/mouse CD11b | M1/70 | FACS | BioLegend |
| Anti-human CD80 | 16-10A1 | FACS | BioLegend |
| Anti-human EpCAM | 9C4 | FACS | BioLegend |
| Anti-human CD56 | HCD56 | FACS | BioLegend |
| Anti-human CD3 | UCHT1 | FACS | BioLegend |
| Anti-human CD19 | SJ25C1 | FACS | BioLegend |
| Anti-human Siglec-5 | 1A5 | FACS | BioLegend |
| Anti-human Siglec-9 | K8 | FACS | BioLegend |
| Anti-human CD45 | 30-F11 | FACS | BioLegend |
| Anti-human F4/80 | BM8 | FACS | BioLegend |
| Anti-human CD24a | M1/69 | FACS | BioLegend |
| Anti-human CSFR1 | AFS98 | Treatment | BioXCell |

[symbol]

indicates data missing or illegible when filed

### Methods:

Statistics. Sample sizes were modeled after those from existing publications regarding in vitro immune killing assays and in vivo tumor growth assays, and an independent statistical method was not used to determine sample size. Statistical tests were performed in Graphpad Prism 8.

Human tumor bulk RNA-sequencing analysis. RNA-sequencing data regarding expression levels for CD24, CD274 (PD-L1), CD47, and B2M from human tumors and matched healthy tissues collected by The Cancer Genome Atlas (TOGA), the Therapeutically Applicable Research to Generate Effective Treatment Program (TARGET), and the Genotype-Tissue Expression Project (GTEX) were downloaded as log 2(Normalized counts+1) values from UCSC with the query “TOGA TARGET GTEX”. Tumor types were filtered for those with ?. 9 individual patients for either tumor or healthy tissues. In instances where there existed both TOGA matched normal tissues and GTEX normal tissues, all normal tissues were combined for analyses. Abbreviations for TOGA studies and number of samples analyzed are listed in Table 1. Survival was performed by stratifying patients into high or low CD24 expression using median expression values, and Kaplan-Meier plots were generated and analyzed using Prism 8. Two dimensional contour plots were generated using Plotly (Plotly Technologies Inc.)

Single-cell RNA-sequencing analysis. Raw files from previously sequenced TNBC (accession 342 PRJNA485423) were downloaded from the NCBI SRA (Karaayvaz et. al 201824). The 1539 single-cell RNA-seq data was aligned to the human genome (GRCh38) using STAR (version 2.5.3a) and gene counts (gene models from ENSEMBL release 82) determined using htseq-count (intersection-nonempty mode, secondary and supplementary alignments ignored, no quality score requirement). The expression matrix was transformed to gene counts per million sequenced reads for each cell. High-quality cells were defined as those that had at least 200,000 cpm and at least 500 genes expressed. This resulted in 1001 cells. Marker genes used in Karaayvaz et. al were used to determine cell types. This was done using UMAP (non-linear dimensionality reduction algorithm) on log-transformed cpm values for the marker genes and labeling each of the five clusters identified based on which cell markers were most expressed (see FIG. 9b). Scatter plots were made using this UMAP transformation with coloring as described in the figure legends.

Cell culture. All cell lines were purchased from ATCC with the exception of the APL1 cells, which were a gift from G. Krampitz (MD Anderson), and the ID8 cells, which were a gift from O. Dorigo. The human NCI-H82 and APL1 cells were cultured in RPMI+GlutaMax (Life Technologies)+10% fetal bovine serum (FBS)+100 U/mL penicillin/streptomycin (Life Technologies). The human MCF-7, Panc1, and U87-GM cell lines were cultured in DMEM+GlutaMax+10% FBS+100 U/mL penicillin/streptomycin. The murine ovarian carcinoma cell line, ID8, was cultured in DMEM+4% FBS+10% Insulin-Transferrin-Selenium (Corning)+100 U/mL penicillin/streptomycin. All cells were cultured in a humidified, 5% CO₂ incubator at 37° C. All cell lines were tested for Mycoplasma.

Generation of MCF-7 and ID8 sub-lines. Parental MCF-7 and ID8 were infected with GFP-luciferase lentivirus in order to generate MCF-7⁻GFP⁻luc⁺ and ID8-GFP-luc⁺ cell lines, respectively. After 48 hours, cells were harvested and sorted by FACS in order to generate pure populations of GFP⁺ cells. The MCF-7/ΔCD24-GFP luc⁺ and ID8/ΔCd24a-GFP⁻luc⁺ sub-lines were generated by electroporating cells with recombinant CRISPR/Cas9 ribonucleoprotein (RNP), as described previously. Briefly, CRISPR/Cas9 guide RNA molecules targeting human CD24 and mouse Cd24a, respectively, were purchased as modified, hybridized RNA molecules (Synthego) and assembled with Cas9-3NLS nuclease (IDT) via incubation at 37° for 45 minutes. Next, 2×10⁶ MCF-7⁻GFP⁻luc⁺ or ID8⁻GFP⁻luc⁺ were harvested, combined with corresponding complexed Cas9/RNP and electroporated using the Lonza Nucleofector IIb using Kit V (VCA-1003). After 48 hours of culture, genetically-modified cells were harvested and purified through at least three successive rounds of FACS sorting in order to generate pure cell lines. Sequences for the guide RNA molecules used are, hCD24 sgRNA: CGGUGCGCGGCGCGUCUAGC, hCD47 sgRNA: AAUAGUAGCUGAGCUGAUCC, and mCd24a sgRNA: AUAUUCUGGU UACCGGGAAA.

In vitro cell proliferation assay. Proliferation of the MCF-7 WT and MCF-7ΔCD24 cell lines was measured with live cell microscopy using an Incucyte (Sartorius). Cells were each plated at ˜10% confluence. Percentage confluence following cell growth was measured as per manufacturer's instructions every 8 h for 64 h.

Neuraminidase treatment and recombinant Siglec binding assay. MCF-7 cells were treated with either neuraminidase (from Vibrio cholerae, Roche) (1×10⁶ cells/100 U/mL) or neuraminidase that was heat inactivated for 15 min at 95° C. prior to incubation for 1 h at 37° C. in serum-free medium, after which reactions were quenched with serum prior to analysis. Recombinant Siglecs (10, 5, and 9) were purchased as human Fc-fusion proteins from R&D Systems. Binding of recombinant Siglecs versus human IgG1 control was assayed at a concentration of 1×10⁵ cells/1 mg/mL at 37° C. for 1 h, in the absence of EDTA. Cells were stained with a fluorescently-conjugated anti-human Fc antibody (Biolegend) to enable the measurement of recombinant Siglec binding by flow cytometry.

Macrophage generation and stimulation. Primary human donor-derived macrophages were generated as described previously. Briefly, leukocyte reduction system (LRS) chambers from anonymous donors were obtained from the Stanford Blood Center. Peripheral monocytes were purified through successive density gradients using Ficoll (Sigma Aldrich) and Percoll (GE Healthcare). Monocytes were then differentiated into macrophages by 7-9 days of culture in IMDM+10% AB human serum (Life Technologies). Unless otherwise stated, macrophages used for all in vitro phagocytosis assays were stimulated with 50 ng/mL human TGFβ1 (Roche) and 50 ng/mL human IL-10 (Roche) on Days 3-4 of differentiation until use on Days 7-9. IL-4 stimulation was added at a concentration of 20 ng/mL on Days 3-4 of differentiation until use on Days 7-9.

Human macrophage knockouts. Genetic knockouts in primary human donor-derived macrophages were performed as described previously. Briefly, sgRNA molecules targeting the first exon of SIGLEC10 were purchased from Synthego as modified, hybridized RNA molecules. The SIGLEC10 sgRNA sequence used is: AGAAUCUCCCAUCCAUAGCC. Mature (day 7) donor-derived macrophages were electroporated with Cas9 ribonuclear proteins using the P3 Primary Cell Nucleofection Kit (Lonza V4XP-3024). Macrophages were harvested for analysis and functional studies 72 hours after electroporation. Indel frequencies were quantified using TIDE software as described previously.

Human samples. The Human Immune Monitoring Center Biobank, the Stanford Tissue Bank, Dr. Oliver Dorigo, and Dr. Gerlinde Wernig all received IRB approval from the Stanford University Administrative Panels on Human Subjects Research and complied with all ethical guidelines for human subjects research to obtain patient samples of ovarian cancer and breast cancer, and received informed consent from all patients. Single cell suspensions of solid tumor specimens were achieved by mechanical dissociation using a straight razor, followed by an enzymatic dissociation in 10 mL of RPMI+10 μg/mL DNasel (Sigma Aldrich)+25 μg/mL Liberase (Roche) for 30-60 min at 37° C. with vigorous pipetting every 10 minutes to promote dissociation. After a maximum of 60 min, dissociation reactions were quenched with 4° C. RPMI+10% FBS and filtered through a 100 micron filter and centrifuged at 400 g for 10 min at 4° C. Red blood cells in samples were then lysed by resuspending tumor pellet in 5 mL ACK Lysing Buffer (Thermo Fisher Scientific) for 5 min at RT. Lysis reactions were quenched by the addition of 20 mL RPMI+10% FBS, and samples were centrifuged at 400 g for 10 min at 4° C. Samples were either directly analyzed, or resuspended in Bambanker (Wako Chemicals USA), aliquoted into cryovials and frozen prior to analysis.

FACS of primary human tumor samples. Single cell suspensions of primary human samples were obtained (described above), and frozen samples were thawed for 3-5 min at 37° C., washed with DMEM+10% FBS, and centrifuged at 400 g for 5 min at 4° C. Samples were then resuspended in FACS buffer at a concentration of 1 million cells per mL and blocked with monoclonal antibody to CD16/32 (Trustain fcX, Biolegend) for 10-15 minutes on ice prior to staining with antibody panels. Antibody panels are listed below, with clones, fluorophores, usage purpose, and concentrations used listed in Table 1. Samples were stained for 30 min on ice, and subsequently washed twice with FACS buffer, and resuspended in buffer containing 1 μg/mL DAPI prior to analysis. Fluorescence compensations were performed using single-stained UltraComp eBeads (Affymetrix). Gating for immune markers and DAPI was performed using fluorescence minus one controls, while CD24+ and Siglec-10⁺ gates were drawn based off of appropriate isotype controls (See Extended Data FIG. 2a for gating strategy). Flow cytometry was performed either on a FACSAria II cell sorter (BD Biosciences) or on an LRSFortessa Analyzer (BD Biosciences) and all flow cytometry data reported in this work was analyzed using FlowJo. Human tumor gating schemes were as follows: Human TAMs: DAPI−, EpCAM−, CD14⁺, CD11b⁺; Human Tumor cells: DAPI−, CD14-, EpCAM⁺.

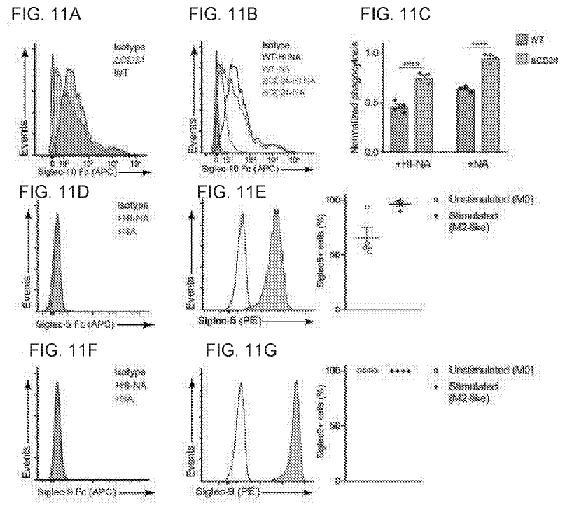

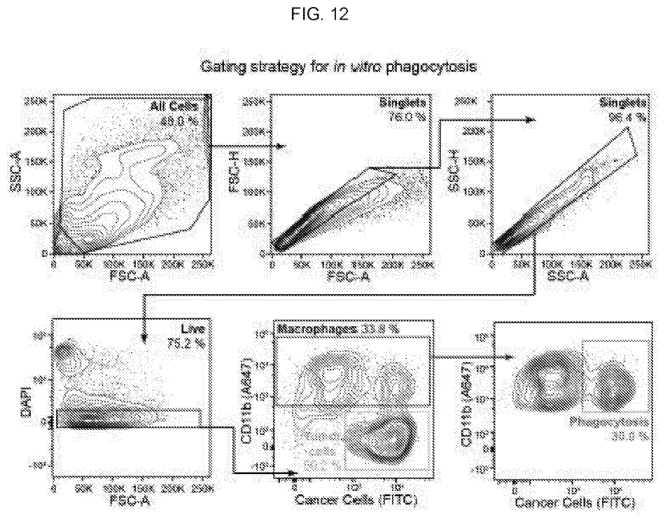

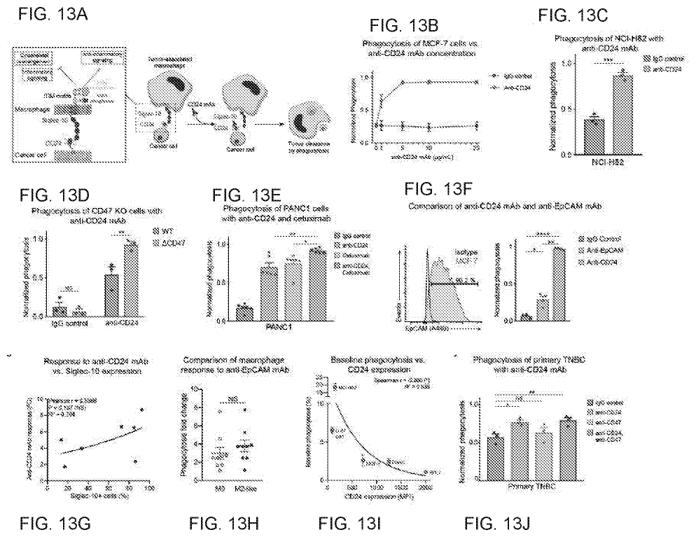

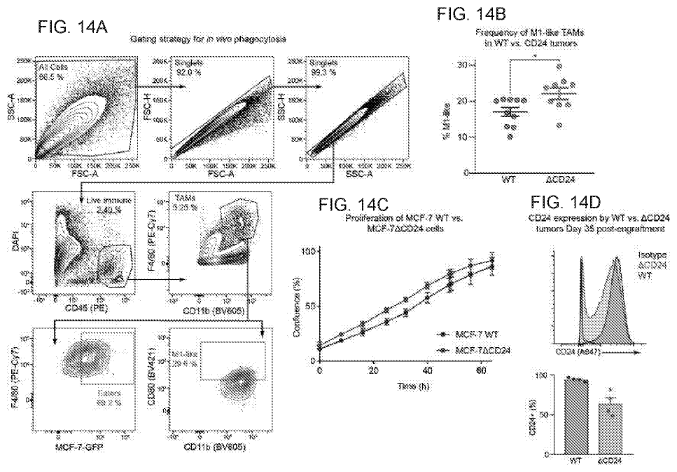

Flow cytometry-based phagocytosis assay. All in vitro phagocytosis assays reported here were performed by co-culture target cells and donor-derived macrophages at a ratio of 100,000 target cells to 50:000 macrophages for 1-2 h in a humidified, 5% CO₂ incubator at 37° C. in ultra-low-attachment 96-well U-bottom plates (Corning) in serum-free IMDM (Life Technologies). Cells with endogenous fluorescence were harvested from plates using TrypLE Express (Life Technologies) prior to co-culture. Cell lines lacking endogenous fluorescence, NCI-H82 and Panc1, were harvested using TrypLE Express and fluorescently labeled with Calcein AM (Invitrogen) by suspending cells in PBS+1:30,000 Calcein AM as per manufacturer instructions for 15 min at 37° C. and washed twice with 40 mL PBS before co-culture. For TNBC primary sample phagocytosis assays, tumors were acquired fresh on the day of resection and dissociated as described above. EpCAM+ tumor cells were purified on an autoMACS pro separator (Miltenyi) by first depleting samples of monocytes using anti-CD14 microbeads (Miltenyi, 1:50) followed by an enrichment with anti-EpCAM microbeads (Miltenyi, 1:50). For primary ovarian cancer ascites assays, ovarian ascites samples were frozen as described above, thawed, and directly labeled with Calcein-AM (Invitrogen) at a concentration of 1:30,000. For primary B cell phagocytosis assays, B cells were enriched from pooled donor PBMC fractions using an autoMACS pro separator (Miltenyi) using anti-CD19 microbeads (Miltenyi, 1:50). For all assays, macrophages were harvested from plates using TrypLE Express. For phagocytosis assays involving treatment with monoclonal antibodies including anti-CD24 (Clone SN3, Novus Biologics) and anti-CD47 (Clone 5F9-G4, acquired from Forty Seven Inc.), all antibodies or appropriate isotype controls were added at a concentration of 10 μg/mL. After co-culture, phagocytosis assays were stopped by placing plates on ice, centrifuged at 400 g for 5 min at 4° C. and stained with A647-labeled anti-CD11b (Clone M1/70, Biolegend) to identify human macrophages. Assays were analyzed by flow cytometry on an LRSFortessa Analyzer (BD Biosciences) or a CytoFLEX (Beckman) both using a high throughput auto-sampler. Phagocytosis was measured as the number of CD11b+, GFP+ macrophages, quantified as a percentage of the total CD11b+ macrophages. Each phagocytosis reaction (independent donor and experimental group) was performed in a minimum of technical triplicate, and outliers were removed using GraphPad Outlier Calculator. In order to account for innate variability in raw phagocytosis levels among donor-derived macrophages, phagocytosis was normalized to the highest technical replicate per donor. All biological replicates indicate independent human macrophage donors. See Table 1 for antibodies and isotype controls used in this study, and FIG. 11 for example gating.

Live-cell microscopy-based phagocytosis assay. Non-fluorescently labeled MCF-7 cells were harvested using TrypLE express and labeled with pHrodo Red, SE (Thermo Fisher Scientific) as per manufacturer instructions at a concentration of 1:30,000 in PBS for 1 h at 37° C., followed by two washes with DMEM+10% FBS+100 U/mL penicillin/streptomycin. Donor-derived macrophages were harvested using TrypLE express and 50,000 macrophages were added to clear, 96-well flat-bottom plates and allowed to adhere for 1 h at 37° C. After macrophage adherence, 100,000 pHrodo-Red-labeled MCF-7 cells+10 μg/mL anti-CD24 antibody (SN3) were added in serum-free IMDM. The plate was centrifuged gently at 50 g for 2 min in order to promote the timely settlement of MCF-7 cells into the same plane as adherent macrophages. Phagocytosis assay plates were then placed in a 37° C. incubator and imaged at 10-20 minute intervals using an Incucyte (Essen). The first image time point (reported as t=0) was generally acquired within 30 minutes after co-culture. Images were acquired using a 20× objective at 800 ms exposures per field. Phagocytosis events were calculated as the number of pHrodo-red+ events per well and values were normalized the maximum number of events measured across technical replicates per donor. Thresholds for calling pHrodo-red+ events were made based off intensity measurements of pHrodo-red-labeled cells lacking any macrophages.

Mice. NOD.Cg-Prkdc_{scid}II2rgₜₘ₁wⱼₗ/SzJ (NSG) mice were obtained from in-house breeding stocks. C57Bl/6J mice were obtained from Jackson Laboratory. All experiments were carried out in accordance with ethical care guidelines set by the Stanford University Administrative Panel on Laboratory Animal Care. Investigators were not blinded for animal studies.

In vivo phagocytosis analysis. For ID8 peritoneal phagocytosis analysis, 4×10⁶, ID8-WT-GFP-luc+ cells or ID8-ΔCd24a-GFP luc+ cells were engrafted into 6-8 week old female NSG mice via intraperitoneal injection of single cell suspensions in PBS. After 7 days, cells were harvested by peritoneal lavage. For MCF-7 xenograft phagocytosis analysis, female NSG mice, 6-10 weeks of age, were engrafted with 4×10⁶ MCF-7-WT-GFP-luc+ cells or MCF-7− MCF-7-ΔCD24-GFP-luc+ cells by injection of single cell suspension in 25% Matrigel Basement Membrane Matrix (Corning)+75% RPMI orthotopically into the mammary fat pad. Tumors were allowed to grow for 28 days after which tumors were resected and dissociated mechanically and enzymatically as described above. Single-cell suspensions of tumors were blocked using anti-CD16/32 (mouse TruStain FcX, BioLegend) for 15 min on ice as described above, prior to staining. Phagocytosis was measured as the percentage of CD11b+, F4/80+ TAMs that were also GFP+(See FIG. 13a for example gating). Mouse TAM gating schemes were as follows: Mouse TAMs: DAPI−, CD45⁺, CD11b⁺, F480⁺; M1-like Mouse TAMs: DAPI−, CD45⁺, CD11b⁺, F480⁺, CD80⁺.

In vivo xenograft tumor growth experiments. Female NSG mice, 6-10 weeks of age, were engrafted with 4×10⁶ MCF-7-WT-GFP-luc+ cells or MCF-7-ΔCD24-GFP-luc+ cells as described above. Tumors were measured using bioluminescence imaging (BLI) beginning 7 days post-engraftment and continuing every 7 days until Day 28. Animals were injected intraperitoneally with D-firefly Luciferin at 140 mg/kg in PBS and images were acquired 10 minutes after luciferin injection using an IVIS Spectrum (Perkin Elmer). Total flux was quantified using Living Image 4.0 software. For survival analyses, animal deaths were reported as the days when primary tumor burden reached 2.5 cm and/or body condition scoring (BCS) values fell below that allowed by our animal protocols.

In vivo macrophage depletion treatment study. Female NSG mice, 6-10 weeks of age were depleted of macrophages as described previously by treatment with 400 μg CSF1R antibody per mouse or PBS (vehicle) (BioXCell, Clone AFS98) three times per week for 18 days prior to engraftment, and throughout the duration of the experiment. Successful tissue resident macrophage depletion was confirmed by flow cytometry prior to tumor engraftment by peritoneal lavage and flow cytometry analysis (FIG. 14). Macrophage-depleted animals or vehicle treated animals were randomized prior to being engrafted with either MCF-7-WT-GFP-luc+ or MCF-7-ΔCD24-GFP-luc+ cells as described above.

Immunocompromised tumor treatment studies. 6-8 week old female NSG mice were engrafted with 4×10₆ MCF-7-WT-GFP-luc+ cells. Day 5 post-engraftment, total flux of all tumors was measured using bioluminescence imaging and engraftment outliers were removed using GraphPad Outlier Calculator. Mice were randomized into treatment groups, receiving either anti-CD24 monoclonal antibody (clone SN3, Creative Diagnostics) or mouse IgG1 isotype control (clone MOPC-21, BioXcell). On day 5 post engraftment, mice received an initial dose of 200 μg and were subsequently treated every other day at a dose of 400 μg for 2 weeks. Bioluminescence imaging was performed throughout the study and after treatment withdrawal in order to assess tumor growth.

In vivo immunocompetent growth experiments. Female C57Bl/6 mice, 6-8 weeks of age were injected intraperitoneally with 1×10₆ ID8-WT tomato-luc+ or ID8-ΔCd24a-tomato-luc+ cells in PBS. Tumor growth was measured by weekly bioluminescence imaging beginning two weeks post-engraftment.

### Example 3

### Antibody: AB1

AB1 antibody is a mouse antibody specifically binds to human CD24. The variable region sequences are provided in the Sequence Listing as SEQ ID NO:1 and SEQ ID NO:5, and the corresponding CDR sequences as SEQ ID NO:2, 3, 4; and SEQ ID NO:6, 7, 8, respectively.

What is claimed is:

1. A method of inducing phagocytosis of a target cell, the method comprising:
contacting a target cell with a macrophage in the presence of an anti-CD24/Siglec10 agent for a period of time sufficient to induce phagocytosis of the target cell by the macrophage.

2. The method of claim 1, wherein the target cell is further contacted with an agent that opsonizes the target cell.

3. The method of claim 1 or claim 2, wherein the agent that opsonizes the target cell is an antibody specific for tumor cell antigen.

4. The method of any of claims 1-3, wherein the target cell is further contacted with an additional agent that enhances phagocytosis.

5. The method of claim 4 wherein the additional agent that enhances phagocytosis is an anti-CD47/SIRPA agent.

6. The method of any of claims 1-5, wherein the target cell is a cancer cell.

7. The method of any of claims 1-5, wherein the target cell is a cell infected with an intracellular pathogen.

8. The method according to any of claims 1-7, wherein the contacting is in vitro or ex vivo.

9. The method according to any of claims 1-7, wherein the contacting is in vivo.

10. The method according to any of claims 1-9, wherein the anti-CD24/Siglec10 agent is an antibody or a binding fragment thereof.

11. The method according to claim 10, wherein the anti-CD24/Siglec10 agent is an antibody specific for Siglec10.

12. The method according to claim 10, wherein the anti-CD24/Siglec10 agent is an antibody specific for CD24.

13. A method of predicting whether an individual is resistant or susceptible to treatment with an anti-CD47/SIRPA agent, the method comprising:
(a) measuring the expression level of CD24 in a biological sample of the individual, wherein the biological sample comprises a cancer cell or a cell harboring an intracellular pathogen, to produce a measured test value;
(b) comparing the measured test value to a control value;
(c) providing a prediction, based on said comparing, as to whether the individual is resistant or susceptible to treatment with an anti-CD47/SIRPA agent; and
(d) treating the patient in accordance with the prediction.

14. The method according to claim 13, wherein said measuring comprises an antibody-based method.

15. The method according to claim 14, wherein the antibody-based method comprises flow cytometry.

16. The method according to any of claims 13-15, wherein the control value is the expression level of CD24 from a cell or population of cells known to exhibit a phenotype of resistance to treatment with an anti-CD47/SIRPA agent.
